(19) United States
(12) Patent Application Publication
(10) Pub. No.: US 2004/0254230 A1
(43) Pub. Date: Dec. 16, 2004

(54) Method for treating ocular hypertension

(76) Inventors: Miller J. Ogidigben, Schwenksville, PA (US); Robert N. Young, Senneville (CA); Kathleen M. Metters, Montreal (CA); Deborah M. Slipetz, Notre Dame Le Perrot (CA)

Correspondence Address: MERCK AND CO INC, P O BOX 2000, RAHWAY, NJ, 070650907

(21) Appl. No.: 10/494,775

(22) PCT Filed: Nov. 27, 2002
(86) PCT No.: PCT/US02/38040

Related U.S. Application Data
(60) Provisional application No. 60/338,117, filed on Dec. 3, 2001.

(51) Int. Cl. A61K 31/557

(52) U.S. Cl. 514/381; 514/424; 514/573

(57) ABSTRACT

This invention relates to potent selective agonists of the EP₄ subtype of prostaglandin E2 receptors, their use or a formulation thereof in the treatment of glaucoma and other conditions that are related to elevated intraocular pressure in the eye of a patient. This invention also relates to the use of such compounds to provide a neuroprotective effect to the eye of mammalian species, particularly humans.

### BACKGROUND OF THE INVENTION

Glaucoma is a degenerative disease of the eye wherein the intraocular pressure is too high to permit normal eye function. As a result, damage may occur to the optic nerve head and result in irreversible loss of visual function. If untreated, glaucoma may eventually lead to blindness. Ocular hypertension, i.e., the condition of elevated intraocular pressure without optic nerve head damage or characteristic glaucomatous visual field defects, is now believed by the majority of ophthalmologists to represent merely the earliest phase in the onset of glaucoma.

Many of the drugs formerly used to treat glaucoma proved unsatisfactory. Early methods of treating glaucoma employed pilocarpine and produced undesirable local effects that made this drug, though valuable, unsatisfactory as a first line drug. More recently, clinicians have noted that many β-adrenergic antagonists are effective in reducing intraocular pressure. While many of these agents are effective for this purpose, there exist some patients with whom this treatment is not effective or not sufficiently effective. Many of these agents also have other characteristics, e.g., membrane stabilizing activity, that become more apparent with increased doses and render them unacceptable for chronic ocular use and can also cause cardiovascular effects.

Agents referred to as carbonic anhydrase inhibitors decrease the formation of aqueous humor by inhibiting the enzyme carbonic anhydrase. While such carbonic anhydrase inhibitors are now used to treat elevated intraocular pressure by systemic and topical routes, current therapies using these agents, particularly those using systemic routes are still not without undesirable effects. Topically effective carbonic anhydrase inhibitors are disclosed in U.S. Pat. Nos. 4,386,098; 4,416,890; 4,426,388; 4,668,697; 4,863,922; 4,797,413; 5,378,703, 5,240,923 and 5,153,192.

Prostaglandins and prostaglandin derivatives are also known to lower intraocular pressure. There are several prostaglandin types, including the A, B, C, D, E, F, G, I and J-Series (EP 0561073 A1). U.S. Pat. No. 4,883,819 to Bito describes the use and synthesis of PGAs, PGBs and PGCs in reducing intraocular pressure. U.S. Pat. No. 4,824,857 to Goh et al. describes the use and synthesis of PGD₂ and derivatives thereof in lowering intraocular pressure including derivatives wherein C-10 is replaced with nitrogen. U.S. Pat. No. 5,001,153 to Ueno et al. describes the use and synthesis of 13,14-dihydro-15-keto prostaglandins and prostaglandin derivatives to lower intraocular pressure. U.S. Pat. No. 4,599,353 describes the use of eicosanoids and eicosanoid derivatives including prostaglandins and prostaglandin inhibitors in lowering intraocular pressure. See also WO 00/38667, WO 99/32441, WO 99/02165, WO 00/38663, WO 01/46140, EP 0855389, JP 2000-1472, U.S. Pat. No. 6,043,275 and WO 00/38690.

Prostaglandin and prostaglandin derivatives are known to lower intraocular pressure by increasing uveoscleral outflow. This is true for both the F type and A type of prostaglandins. This invention is particularly interested in those compounds that lower IOP via the uveoscleral outflow pathway and other mechanisms by which the E series prostaglandins (PGE₂) may facilitate IOP reduction. While the relationship between EP receptor activation and IOP lowering effects is not well understood, there are four recognized subtypes of the EP receptor (EP₁, EP₂, EP₃ and EP₄; J. Lipid Mediators Cell Signaling, Vol. 14, pages 83-87 (1996)). See also J. Ocular Pharmacology, Vol. 4, 1, pages 13-18 (1988); J. Ocular Pharmacology and Therapeutics, Vol. 11, 3, pages 447-454 (1995); J. Lipid Mediators, Vol. 6, pages 545-553 (1993); U.S. Pat. Nos. 5,698,598 and 5,462,968 and Investigative Ophthalmology and Visual Science, Vol. 31, 12, pages 2560-2567 (1990). Of particular interest to this invention are compounds, which are agonist of the EP₄ subtype receptor.

A problem with using prostaglandins or derivatives thereof to lower intraocular pressure is that these compounds often induce an initial increase in intraocular pressure, can change the color of eye pigmentation and cause proliferation of some tissues surrounding the eye.

As can be seen, there are several current therapies for treating glaucoma and elevated intraocular pressure, but the efficacy and the side effect profiles of these agents are not ideal. Therefore, there still exist the need for new and effective therapies with little or no side effects.

### SUMMARY OF THE INVENTION

This invention relates to the use of potent selective agonists of the EP₄ subtype of prostaglandin E2 receptors in the treatment of glaucoma and other conditions which are related to elevated intraocular pressure in the eye of a patient. This invention also relates to the use of such compounds to provide a neuroprotective effect to the eye of mammalian species and/or treat dry eye in mammals, particularly humans. More particularly this invention relates to the treatment of glaucoma and/or ocular hypertension (elevated intraocular pressure) using compounds having the structural formula Ia, Ib, Ic, Id, Ie or If:

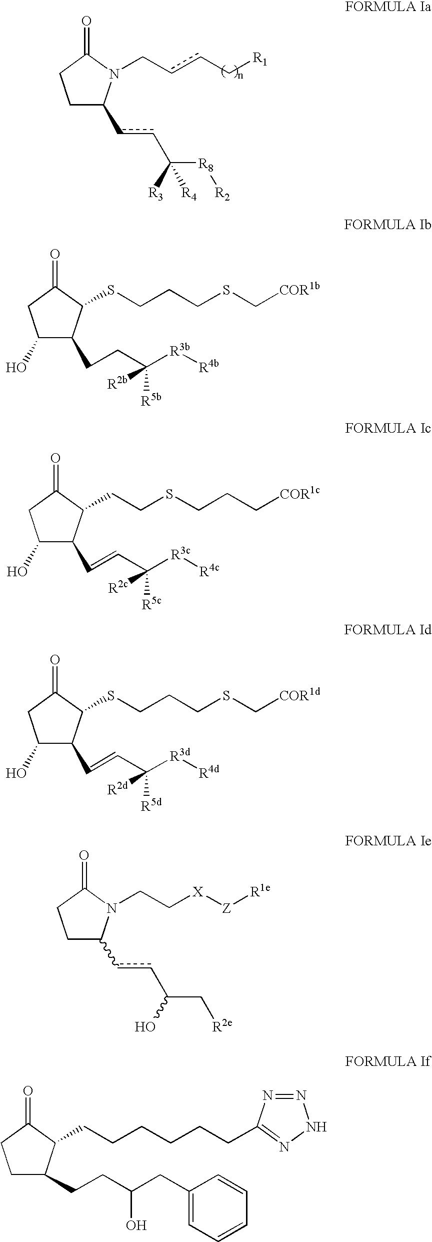

or a pharmaceutically acceptable salt, cyclodextrin clathrate, enantiomer, diastereomer or mixture thereof:

wherein,

R₁ represents COOR₅, CONHR₆ or tetrazol-5-yl;

R₃ and R₄ are each independently hydrogen, hydroxy or C₁₋₃ alkyl;

R₂ represents hydrogen, α-thienyl, phenyl or phenoxy, wherein said phenyl and phenoxy are optionally substituted with 1-3 substituents selected from chloro, fluoro, phenyl, methoxy, trifluoromethyl or C₁₋₃ alkyl;

[symbol] represents a single or double bond;

n is 0to 3;

R₅ represents hydrogen, C₁₋₅ alkyl, phenyl or p-biphenyl;

R₆ represents COR₇ or SO₂R₇;

R₇ represents phenyl or C₁₋₅ alkyl;

R₈ represents C₃₋₆ cycloalkyl or C₁₋₆ alkyl, wherein said cycloalkyl and alkyl groups are optionally substituted with one or two C₁₋₆ alkyl groups;

R^{1b} represents hydroxy, C₁₋₆ alkyloxy or NR^{6b}R^{7b}, wherin R^{6b} and R^{7b} are each independently hydrogen or C₁₋₆ alkyl;

R^{2b} represents hydrogen or hydroxy;

R^{3b} represents a single bond or C₁₋₆ alkylene;

R^{4b} represents

(i) C₁₋₈ alkyl, C₂₋₈ alkenyl, C₂₋₈ alkynyl, phenyl or cycloalkyl, wherein said alkyl, alkenyl, alkynyl are optionally substituted with phenyl, cycloalkyl, OH, C₁₋₆ alkyloxy or halogen;

(ii) Phenyloxy or C₃₋₇ cycloalkyloxy;

(iii) Furyl, furyloxy, thienyl, thienyloxy, naphthyl, naphthyloxy, phthalanyl or phthalanyloxy;

(iv) Phenyl, phenyloxy, C₃₋₇ cycloalkyl or C₃₋₇ cycloalkyloxy, wherein said phenyl, phenyloxy, cycloalkyl and cycloalkyloxy groups are optionally substituted with 1-3 substituents selected from C₁₋₆ alkyl, C₂₋₆ alkenyl, C₂₋₆ alkynyl, C₁₋₆ alkyloxy, C₁₋₆ alkyloxy-C₁₋₆ alkyl, C₁₋₆ alkyloxy-C₁₋₆ alkyloxy, C₁₋₆ alkenyloxy-C₁₋₆ alkyl, C₁₋₆ alkyl substituted by 1-3 hydroxy or halogen groups, C₁₋₆ alkylthio, C₁₋₆ alkylthio-C₁₋₆ alkyl, C₁₋₆ alkylthio-C₁₋₆ alkyloxy, C₁₋₆ alkenylthio-C₁₋₆ alkyl, C₁₋₆ alkylsulfonyl, halogen, trihalomethyl, cyano, nitro, amino, OH, C₃₋₇ cycloalkyl, C₃₋₇ cycloalkyloxy, C₃₋₇ cycloalkyl-C₁₋₆ alkyl, C₃₋₇ cycloalkyloxy-C₁₋₆ alkyl, phenyl, phenyloxy, phenyl-C₁₋₆ alkyl, phenyl-C₂₋₆ alkenyl, phenyl-C₂₋₆ alkynyl, phenyloxy-C₁₋₆ alkyl, phenyloxy-C₂₋₆ alkenyl, phenyloxy-C₂₋₆ alkynyl, furyl, furyloxy, furyl-C₁₋₆ alkyl, furyloxy-C₁₋₆ alkyl, thienyl, thienyloxy, thienyl-C₁₋₆ alkyl or thienyloxy-C₁₋₆ alkyl, wherein said phenyl, furyl, thienyl or cycloalkyl are optionally substituted with 1-3 groups selected from C₁₋₆ alkyl, C₁₋₆ alkyloxy, C₁₋₆alkyloxy-C₁₋₆ alkyl, nitro, halogen, trihalomethyl, amino or hydroxy; or

(v) Furyl, furyloxy, thienyl, thienyloxy, naphthyl, naphthyloxy, phthalanyl or phthalanyloxy optionally substituted with 1-3 groups selected from C₁₋₆ alkyl, C₂₋₆ alkenyl, C₂₋₆ alkynyl, C₁₋₆ akyloxy, C₁₋₆ alkyloxy-C₁₋₆ alkyl, C₁₋₆ alkyloxy-C₁₋₆ alkyloxy, C₂₋₆ alkenyloxy-C₁₋₆ alkyl, C₁₋₆ alkyl substituted with 1-3 groups of hydroxy or halogen, C₁₋₆ alkylthio, C₁₋₆ alkylthio-C₁₋₆ alkyl, C₁₋₆ alkylthio-C₁₋₆ alkyloxy, C₁₋₆ alkenylthio-C₁₋₆ alkyl, C₁₋₆ alkylsulfonyl, halogen, trihalomethyl, cyano, nitro, amino, hydroxy, C₃₋₇ cycloalkyl, C₃₋₇ cycloalkyloxy, C₃₋₇ cycloalkyl-C₁₋₆ alkyl, C₃₋₇ cycloalkyloxy-C₁₋₆ alkyl, phenyl, phenyloxy, phenyl-C₁₋₆ alkyl, phenyl-C₂₋₆ alkenyl, phenyl-C₂₋₆ alkynyl, phenyloxy-C₁₋₆ alkyl, phenyloxy-C₂₋₆ alkenyl, phenyloxy-C₂₋₆ alkynyl, furyl, furyloxy, furyl-C₁₋₆ alkyl, furyloxy-C₁₋₆ alkyl, thienyl, thienyloxy, thienyl-C₁₋₆ alkyl and thienyloxy-C₁₋₆ alkyl, said phenyl, furyl, thienyl or cycloalkyl optionally substituted with 1-3 groups selected from C₁₋₆ alkyl, C₁₋₆ alkyloxy, C₁₋₆alkyloxy-C₁₋₆ alkyl, nitro, halogen, trihalomethyl, amino and hydroxy;

R^{5b} represents hydrogen or C 1-6 alkyl;

R^{1c} represents hydroxy, C₁₋₆ alkyloxy or NR^{6c}R^{7c}, wherin R^{6c} and R^{7c} are each independently hydrogen or C₁₋₆ alkyl;

R^{2c} represents hydrogen or hydroxy;

R^{3c} represents a single bond or C₁₋₆ alkylene;

R^{4c} represents

(i) C₁₋₈ alkyl, C₂₋₈ alkenyl, C₂₋₈ alkynyl, phenyl or cycloalkyl, wherein said alkyl, alkenyl, alkynyl are optionally substituted with phenyl, cycloalkyl, OH, C₁₋₆ alkyloxy or halogen;

(ii) Phenyloxy or C₃₋₇ cycloalkyloxy;

(iii) Furyl, furyloxy, thienyl, thienyloxy, naphthyl, naphthyloxy, phthalanyl or phthalanyloxy;

(iv) Phenyl, phenyloxy, C₃₋₇ cycloalkyl or C₃₋₇ cycloalkyloxy, wherein said phenyl, phenyloxy, cycloalkyl and cycloalkyloxy groups are optionally substituted with 1-3 substituents selected from C₁₋₆ alkyl, C₂₋₆ alkenyl, C₂₋₆ alkynyl, C₁₋₆ alkyloxy, C₁₋₆ alkyloxy-C₁₋₆ alkyl, C₁₋₆ alkyloxy-C₁₋₆ alkyloxy, C₁₋₆ alkenyloxy-C₁₋₆ alkyl, C₁₋₆ alkyl substituted by 1-3 hydroxy or halogen groups, C₁₋₆ alkylthio, C₁₋₆ alkylthio-C₁₋₆ alkyl, C₁₋₆ alkylthio-C₁₋₆ alkyloxy, C₁₋₆ alkenylthio-C₁₋₆ alkyl, C₁₋₆ alkylsulfonyl, halogen, trihalomethyl, cyano, nitro, amino, OH, C₃₋₇ cycloalkyl, C₃₋₇ cycloalkyloxy, C₃₋₇ cycloalkyl-C₁₋₆ alkyl, C₃₋₇ cycloalkyloxy-C₁₋₆ alkyl, phenyl, phenyloxy, phenyl-C₁₋₆ alkyl, phenyl-C₂₋₆ alkenyl, phenyl-C₂₋₆ alkynyl, phenyloxy-C₁₋₆ alkyl, phenyloxy-C₂₋₆ alkenyl, phenyloxy-C₂₋₆ alkynyl, furyl, furyloxy, furyl-C₁₋₆ alkyl, furyloxy-C₁₋₆ alkyl, thienyl, thienyloxy, thienyl-C₁₋₆ alkyl or thienyloxy-C₁₋₆ alkyl, wherein said phenyl, furyl, thienyl or cycloalkyl are optionally substituted with 1-3 groups selected from C₁₋₆ alkyl, C₁₋₆ alkyloxy, C₁₋₆alkyloxy-C₁₋₆ alkyl, nitro, halogen, trihalomethyl, amino or hydroxy; or

(v) Furyl, furyloxy, thienyl, thienyloxy, naphthyl, naphthyloxy, phthalanyl or phthalanyloxy optionally substituted with 1-3 groups selected from C₁₋₆ alkyl, C₂₋₆ alkenyl, C₂₋₆ alkynyl, C₁₋₆ akyloxy, C₁₋₆ alkyloxy-C₁₋₆ alkyl, C₁₋₆ alkyloxy-C₁₋₆ alkyloxy, C₂₋₆ alkenyloxy-C₁₋₆ alkyl, C₁₋₆ alkyl substituted with 1-3 groups of hydroxy or halogen, C₁₋₆ alkylthio, C₁₋₆ alkylthio-C₁₋₆ alkyl, C₁₋₆ alkylthio-C₁₋₆ alkyloxy, C₁₋₆ alkenylthio-C₁₋₆ alkyl, C₁₋₆ alkylsulfonyl, halogen, trihalomethyl, cyano, nitro, amino, hydroxy, C₃₋₇ cycloalkyl, C₃₋₇ cycloalkyloxy, C₃₋₇ cycloalkyl-C₁₋₆ alkyl, C₃₋₇ cycloalkyloxy-C₁₋₆ alkyl, phenyl, phenyloxy, phenyl-C₁₋₆ alkyl, phenyl-C₂₋₆ alkenyl, phenyl-C₂₋₆ alkynyl, phenyloxy-C₁₋₆ alkyl, phenyloxy-C₂₋₆ alkenyl, phenyloxy-C₂₋₆ alkynyl, furyl, furyloxy, furyl-C₁₋₆ alkyl, furyloxy-C₁₋₆ alkyl, thienyl, thienyloxy, thienyl-C₁₋₆ alkyl and thienyloxy-C₁₋₆ alkyl, said phenyl, furyl, thienyl or cycloalkyl optionally substituted with 1-3 groups selected from C₁₋₆ alkyl, C₁₋₆ alkyloxy, C₁₋₆alkyloxy-C₁₋₆ alkyl, nitro, halogen, trihalomethyl, amino and hydroxy;

R^{5c} represents hydrogen, hydroxy or C 1-6 alkyl;

R^{1d} represents hydroxy, C₁₋₆ alkyloxy or NR^{6d}R^{7d}, wherin R^{6d} and R^{7d} are each independently hydrogen or C₁₋₆ alkyl;

R^{2d} represents hydrogen or hydroxy;

R^{3d} represents a single bond or C₁₋₆ alkylene;

R^{4d} represents

(i) C₁₋₈ alkyl, C₂₋₈ alkenyl, C₂₋₈ alkynyl, phenyl or cycloalkyl, wherein said alkyl, alkenyl, alkynyl are optionally substituted with phenyl, cycloalkyl, OH, C₁₋₆ alkyloxy or halogen;

(ii) Phenyloxy or C₃₋₇ cycloalkyloxy;

(iii) Furyl, furyloxy, thienyl, thienyloxy, naphthyl, naphthyloxy, phthalanyl or phthalanyloxy;

(iv) Phenyl, phenyloxy, C₃₋₇ cycloalkyl or C₃₋₇ cycloalkyloxy, wherein said phenyl, phenyloxy, cycloalkyl and cycloalkyloxy groups are optionally substituted with 1-3 substituents selected from C₁₋₆ alkyl, C₂₋₆ alkenyl, C₂₋₆ alkynyl, C₁₋₆ alkyloxy, C₁₋₆ alkyloxy-C₁₋₆ alkyl, C₁₋₆ alkyloxy-C₁₋₆ alkyloxy, C₁₋₆ alkenyloxy-C₁₋₆ alkyl, C₁₋₆ alkyl substituted by 1-3 hydroxy or halogen groups, C₁₋₆ alkylthio, C₁₋₆ alkylthio-C₁₋₆ alkyl, C₁₋₆ alkylthio-C₁₋₆ alkyloxy, C₁₋₆ alkenylthio-C₁₋₆ alkyl, C₁₋₆ alkylsulfonyl, halogen, trihalomethyl, cyano, nitro, amino, OH, C₃₋₇ cycloalkyl, C₃₋₇ cycloalkyloxy, C₃₋₇ cycloalkyl-C₁₋₆ alkyl, C₃₋₇ cycloalkyloxy-C₁₋₆ alkyl, phenyl, phenyloxy, phenyl-C₁₋₆ alkyl, phenyl-C₂₋₆ alkenyl, phenyl-C₂₋₆ alkynyl, phenyloxy-C₁₋₆ alkyl, phenyloxy-C₂₋₆ alkenyl, phenyloxy-C₂₋₆ alkynyl, furyl, furyloxy, furyl-C₁₋₆ alkyl, furyloxy-C₁₋₆ alkyl, thienyl, thienyloxy, thienyl-C₁₋₆ alkyl or thienyloxy-C₁₋₆ alkyl, wherein said phenyl, furyl, thienyl or cycloalkyl are optionally substituted with 1-3 groups selected from C₁₋₆ alkyl, C₁₋₆ alkyloxy, C₁₋₆alkyloxy-C₁₋₆ alkyl, nitro, halogen, trihalomethyl, amino or hydroxy; or

(v) Furyl, furyloxy, thienyl, thienyloxy, naphthyl, naphthyloxy, phthalanyl or phthalanyloxy optionally substituted with 1-3 groups selected from C₁₋₆ alkyl, C₂₋₆ alkenyl, C₂₋₆ alkynyl, C₁₋₆ akyloxy, C₁₋₆ alkyloxy-C₁₋₆ alkyl, C₁₋₆ alkyloxy-C₁₋₆ alkyloxy, C₂₋₆ alkenyloxy-C₁₋₆ alkyl, C₁₋₆ alkyl substituted with 1-3 groups of hydroxy or halogen, C₁₋₆ alkylthio, C₁₋₆ alkylthio-C₁₋₆ alkyl, C₁₋₆ alkylthio-C₁₋₆ alkyloxy, C₁₋₆ alkenylthio-C₁₋₆ alkyl, C₁₋₆ alkylsulfonyl, halogen, trihalomethyl, cyano, nitro, amino, hydroxy, C₃₋₇ cycloalkyl, C₃₋₇ cycloalkyloxy, C₃₋₇ cycloalkyl-C₁₋₆ alkyl, C₃₋₇ cycloalkyloxy-C₁₋₆ alkyl, phenyl, phenyloxy, phenyl-C₁₋₆ alkyl, phenyl-C₂₋₆ alkenyl, phenyl-C₂₋₆ alkynyl, phenyloxy-C₁₋₆ alkyl, phenyloxy-C₂₋₆ alkenyl, phenyloxy-C₂₋₆ alkynyl, furyl, furyloxy, furyl-C₁₋₆ alkyl, furyloxy-C₁₋₆ alkyl, thienyl, thienyloxy, thienyl-C₁₋₆ alkyl and thienyloxy-C₁₋₆ alkyl, said phenyl, furyl, thienyl or cycloalkyl optionally substituted with 1-3 groups selected from C₁₋₆ alkyl, C₁₋₆ alkyloxy, C₁₋₆alkyloxy-C₁₋₆ alkyl, nitro, halogen, trihalomethyl, amino and hydroxy;

R^{5d} represents hydrogen, hydroxy or C 1-6 alkyl;

R^{1e} represents carboxyl, (C₃-C₄)alkoxylcarbonyl or tetrazolyl;

R^{2e} represents —Ar, or —Ar¹-V-Ar², wherein V is a bond, —O—, —OCH₂— or —CH₂O—;

X represents —CH₂— or 0;

Z represents —(CH₂)₃—, thienyl, thiazolyl, or phenyl;

Ar represents a partially saturated, fully saturated or fully unsaturated five to eight membered ring optionally having one to four heteroatoms selected independently from oxygen, sulfur and nitrogen, or a bicyclic ring consisting of two fused independently partially saturated, fully saturated or fully unsaturated five or six membered rings, taken independently, optionally having one to four heteroatoms selected independently from nitrogen, sulfur and oxygen, said partially or fully saturated ring or bicyclic ring optionally having one or two oxo groups substituted on carbon or one or two oxo groups substituted on sulfur; and

Ar¹ and Ar² each independently represent a partially saturated, fully saturated or fully unsaturated five to eight membered ring optionally having one to four heteroatoms selected independently from oxygen, sulfur and nitrogen, said partially or fully saturated ring optionally having one or two oxo groups substituted on carbon or one or two oxo groups substituted on sulfur;

wherein said Ar moiety is optionally substituted on carbon or nitrogen, on one ring if the moiety is monocyclic, or on one or both rings if the moiety is bicyclic, with up to three substituents per ring each independently selected from hydroxy, halo, carboxy, (C₁-C₇)alkoxy, (C₁-C₄)alkoxy(C₁-C₄)alkyl, (C₁-C₇)alkyl, (C₂-C₇)alkenyl, (C₃-C₇)cycloalkyl, (C₃-C₇)cycloalkyl(C₁-C₄)alkyl, (C₃-C₇)cycloalkyl(C₁-C₄)alkanoyl, formyl, (C₁-C₈)alkanoyl, (C₁-C₆)alkanoyl(C₁-C₆)alkyl, (C₁-C₄)alkanoylamino, (C₁-C₄)alkoxycarbonylamino, hydroxysulfonyl, aminocarbonylamino or mono-N-, di-N,N-, di-N,N′- or tri-N,N,N′-(C₁-C₄)alkyl substituted aminocarbonylamino, sulfonamido, (C₁-C₄)alkylsulfonamido, amino, mono-N- or di-N,N-(C₁-C₄)alkylamino, carbamoyl, mono-N- or di-N,N-(C₁-C₄)alkylcarbamoyl, cyano, thiol, (C₁-C₆)alkylthio, (C₁-C₆)alkylsulfinyl, (C₁-C₄)alkylsulfonyl and mono-N- or di-N,N-(C₁-C₄)alkylaminosulfinyl, wherein said alkyl and alkoxy substituents in the definition of Ar are optionally substituted on carbon with up to three fluoro; and

wherein said Ar¹ and Ar² moieties are independently optionally substituted on carbon or nitrogen with up to three substituents each independently selected from hydroxy, halo, carboxy, (C₁-C₇)alkoxy, (C₁-C₄)alkoxy(C₁-C₄)alkyl, (C₁-C₇)alkyl, (C₂-C₇)alkenyl, (C₃-C₇)cycloalkyl, (C₃-C₇)cycloalkyl(C₁-C₄)alkyl, (C₃-C₇)cycloalkyl(C₁-C₄)alkanoyl, formyl, (C₁-C₈)alkanoyl, (C₁-C₆)alkanoyl(C₁-C₆)alkyl, (C₁-C₄)alkanoylamino, (C₁-C₄)alkoxycarbonylamino, hydroxysulfonyl, aminocarbonylamino or mono-N-, di-N,N-, di-N,N′- or tri-N,N,N′-(C₁-C₄)alkyl substituted aminocarbonylamino, sulfonamido, (C₁-C₄)alkylsulfonamido, amino, mono-N- or di-N,N-(C₁-C₄)alkylamino, carbamoyl, mono-N- or di-N,N-(C₁-C₄)alkylcarbamoyl, cyano, thiol, (C₁-C₆)alkylthio, (C₁-C₆)alkylsulfinyl, (C₁-C₄)alkylsulfonyl and mono-N- or di-N,N-(C₁-C₄)alkylaminosulfinyl, wherein said alkyl and alkoxy substituents in the definition of Ar¹ and Ar² are optionally substituted on carbon with up to three fluoro.

This and other aspects of the invention will be realized upon inspection of the invention as a whole.

### DETAILED DESCRIPTION OF THE INVENTION

The present invention is directed to a method for decreasing elevated intraocular pressure or treating glaucoma by administration, preferably topical or intra-camaral, of a composition containing an EP₄ agonist of Formula Ia, Ib, Ic, Id, Ie or If and a pharmaceutically acceptable carrier.

An embodiment of the invention of formula Ia is realized when n is three and all other variables are as originally described.

An embodiment of the invention of formula Ia is realized when R₃ and R₄ are each independently hydrogen or hydroxy and all other variables are as originally described.

An embodiment of the invention of formula Ia is realized when R₁ is COOH and all other variables are as originally described.

An embodiment of the invention of formula Ia is realized when R₁ is tetrazol-5-yl and all other variables are as originally described.

Another embodiment of the invention of formula Ia is realized when R₁ is tetrazol-5-yl, R₂ is phenyl and all other variables are as originally described.

An embodiment of the invention of formula Ia is realized when R₈ is CH₂ and all other variables are as originally described.

An embodiment of the invention of formula Ib is realized when R^{1b} is hydroxy and all other variables are as originally described.

Another embodiment of the invention of formula Ib is realized when R^{3b} is a single bond and all other variables are as originally described.

Still another embodiment of the invention of formula Ib is realized when R^{1b} is C₁₋₆ alkyloxy and all other variables are as originally described.

Yet another embodiment of the invention of formula Ib is realized when R^{1b} is NR^{6b}R^{7b} wherein R^{6b} and R^{7b} are independently hydrogen or C₁₋₆ alkyl and all other variables are as originally described.

Another embodiment of the invention of formula Ib is realized when R^{3b} is a single bond and R^{4b} is C₁₋₈ alkyl, C₂₋₈ alkenyl, phenyl, C₃₋₇ cycloalkyl or C₂₋₈ alkynyl, said alkyl, alkenyl and alkynyl optionally substituted with C₁₋₆ alkyloxy or halogen.

Another embodiment of the invention of formula Ib is realized when R^{4b} is C₁₋₈ alkyl, C₂₋₈ alkenyl, or C₂₋₈ alkynyl, said alkyl, alkenyl and alkynyl optionally substituted with C₁₋₆ alkyloxy or halogen.

Another embodiment of the invention of formula Ib is realized when R^{4b} is phenyloxy or C₃₋₇ cycloalkyloxy.

An embodiment of the invention of formula Ic is realized when R^{1c} is C₁₋₆ alkyloxy.

Another embodiment of the invention of formula Ic is realized when R^{2c} is hydrogen.

Another embodiment of the invention of formula Ic is realized when R^{5c} is hydroxy.

Another embodiment of the invention of formula Ic is realized when R^{4c} is phenyl which is optionally substituted with 1-3 substituents selected from C₁₋₆ alkyl, C₂₋₆ alkenyl, C₂₋₆ alkynyl, C₁₋₆ alkyloxy, C₁₋₆ alkyloxy-C₁₋₆ alkyl, C₁₋₆ alkyloxy-C₁₋₆ alkyloxy, C₁₋₆ alkenyloxy-C₁₋₆ alkyl, C₁₋₆ alkyl substituted by 1-3 hydroxy or halogen groups, C₁₋₆ alkylthio, C₁₋₆ alkylthio-C₁₋₆ alkyl, C₁₋₆ alkylthio-C₁₋₆ alkyloxy, C₁₋₆ alkenylthio-C₁₋₆ alkyl, C₁₋₆ alkylsulfonyl, halogen, trihalomethyl, cyano, nitro, amino, OH, C₃₋₇ cycloalkyl, C₃₋₇ cycloalkyloxy, C₃₋₇ cycloalkyl-C₁₋₆ alkyl, C₃₋₇ cycloalkyloxy-C₁₋₆ alkyl, phenyl, phenyloxy, phenyl-C₁₋₆ alkyl, phenyl-C₂₋₆ alkenyl, phenyl-C₂₋₆ alkynyl, phenyloxy-C₁₋₆ alkyl, phenyloxy-C₂₋₆ alkenyl, phenyloxy-C₂₋₆ alkynyl, furyl, furyloxy, furyl-C₁₋₆ alkyl, furyloxy-C₁₋₆ alkyl, thienyl, thienyloxy, thienyl-C₁₋₆ alkyl or thienyloxy-C₁₋₆ alkyl, wherein said phenyl, furyl, thienyl or cycloalkyl are optionally substituted with 1-3 groups selected from C₁₋₆ alkyl, C₁₋₆ alkyloxy, C₁₋₆alkyloxy-C₁₋₆ alkyl, nitro, halogen, trihalomethyl, amino or hydroxy.

An embodiment of the invention of formula Id is realized when R^{1d} is C₁₋₆ hydroxy.

Another embodiment of the invention of formula Id is realized when R^{2d} is hydrogen.

Another embodiment of the invention of formula Id is realized when R^{5d} is hydroxy.

Another embodiment of the invention of formula Id is realized when R^{4d} is phenyl which is optionally substituted with 1-3 substituents selected from C₁₋₆ alkyl, C₂₋₆ alkenyl, C₂₋₆ alkynyl, C₁₋₆ alkyloxy, C₁₋₆ alkyloxy-C₁₋₆ alkyl, C₁₋₆ alkyloxy-C₁₋₆ alkyloxy, C₁₋₆ alkenyloxy-C₁₋₆ alkyl, C₁₋₆ alkyl substituted by 1-3 hydroxy or halogen groups, C₁₋₆ alkylthio, C₁₋₆ alkylthio-C₁₋₆ alkyl, C₁₋₆ alkylthio-C₁₋₆ alkyloxy, C₁₋₆ alkenylthio-C₁₋₆ alkyl, C₁₋₆ alkylsulfonyl, halogen, trihalomethyl, cyano, nitro, amino, OH, C₃₋₇ cycloalkyl, C₃₋₇ cycloalkyloxy, C₃₋₇ cycloalkyl-C₁₋₆ alkyl, C₃₋₇ cycloalkyloxy-C₁₋₆ alkyl, phenyl, phenyloxy, phenyl-C₁₋₆ alkyl, phenyl-C₂₋₆ alkenyl, phenyl-C₂₋₆ alkynyl, phenyloxy-C₁₋₆ alkyl, phenyloxy-C₂₋₆ alkenyl, phenyloxy-C₂₋₆ alkynyl, furyl, furyloxy, furyl-C₁₋₆ alkyl, furyloxy-C₁₋₆ alkyl, thienyl, thienyloxy, thienyl-C₁₋₆ alkyl or thienyloxy-C₁₋₆ alkyl, wherein said phenyl, furyl, thienyl or cycloalkyl are optionally substituted with 1-3 groups selected from C₁₋₆ alkyl, C₁₋₆ alkyloxy, C₁₋₆alkyloxy-C₁₋₆ alkyl, nitro, halogen, trihalomethyl, amino or hydroxy.

An embodiment of the invention of formula Ie is realized when X is —CH₂—, Z is —(CH₂)₃—,

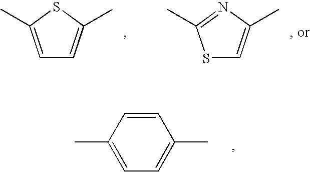

and R^{2e} is Ar.

Also embodying the invention are potent selective agonists of the EP₄ subtype of prostaglandin E2 receptors useful in the treatment of glaucoma and other conditions which are related to elevated intraocular pressure in the eye of a patient. This invention also relates to the use of such compounds to provide a neuroprotective effect to the eye of mammalian species and/or treat dry eye in mammals, particularly humans. More particularly this invention relates to the treatment of glaucoma and/or ocular hypertension (elevated intraocular pressure) using compounds having the structural formula Ia or Ib:

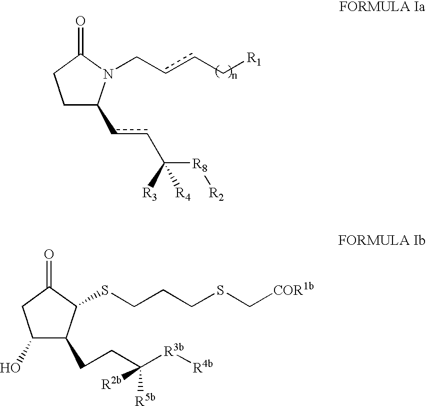

wherein R₁ represents COOR₅, CONHR₆ or tetrazol-5-yl;

R₃ and R₄ are each independently hydrogen or hydroxy;

R₂ represents α-thienyl, phenyl or phenoxy, wherein said phenyl and phenoxy are optionally substituted with 1-3 substituents selected from chloro, fluoro, phenyl, methoxy, trifluoromethyl or C₁₋₃ alkyl;

[symbol] represents a single or double bond;

n is 0 to 3;

R₅ represents hydrogen, C₁₋₅ alkyl, phenyl or p-biphenyl;

R₆ represents COR₇ or SO₂R₇;

R₇ represents phenyl or C₁₋₅ alkyl;

R₈ represents CH₂;

R^{1b} represents hydroxy, C₁₋₆ alkyloxy or NR^{6b}R^{7b}, wherin R^{6b} and R^{7b} are each independently hydrogen or C₁₋₆ alkyl;

R^{2b} represents hydrogen or hydroxy;

R^{3b} represents a single bond or C₁₋₆ alkylene;

R^{4b} represents

(i) C₁₋₈ alkyl, C₂₋₈ alkenyl, C₂₋₈ alkynyl, phenyl or cycloalkyl, wherein said alkyl, alkenyl, alkynyl are optionally substituted with phenyl, cycloalkyl, OH, C₁₋₆ alkyloxy or halogen;

(ii) Phenyloxy or C₃₋₇ cycloalkyloxy;

(iii) Furyl, furyloxy, thienyl, thienyloxy, naphthyl, naphthyloxy, phthalanyl or phthalanyloxy;

(iv) Phenyl, phenyloxy, C₃₋₇ cycloalkyl or C₃₋₇ cycloalkyloxy, wherein said phenyl, phenyloxy, cycloalkyl and cycloalkyloxy groups are optionally substituted with 1-3 substituents selected from C₁₋₆ alkyl, C₂₋₆ alkenyl, C₂₋₆ alkynyl, C₁₋₆ alkyloxy, C₁₋₆ alkyloxy-C₁₋₆ alkyl, C₁₋₆ alkyloxy-C₁₋₆ alkyloxy, C₁₋₆ alkenyloxy-C₁₋₆ alkyl, C₁₋₆ alkyl substituted by 1-3 hydroxy or halogen groups, C₁₋₆ alkylthio, C₁₋₆ alkylthio-C₁₋₆ alkyl, C₁₋₆ alkylthio-C₁₋₆ alkyloxy, C₁₋₆ alkenylthio-C₁₋₆ alkyl, C₁₋₆ alkylsulfonyl, halogen, trihalomethyl, cyano, nitro, amino, OH, C₃₋₇ cycloalkyl, C₃₋₇ cycloalkyloxy, C₃₋₇ cycloalkyl-C₁₋₆ alkyl, C₃₋₇ cycloalkyloxy-C₁₋₆ alkyl, phenyl, phenyloxy, phenyl-C₁₋₆ alkyl, phenyl-C₂₋₆ alkenyl, phenyl-C₂₋₆ alkynyl, phenyloxy-C₁₋₆ alkyl, phenyloxy-C₂₋₆ alkenyl, phenyloxy-C₂₋₆ alkynyl, furyl, furyloxy, furyl-C₁₋₆ alkyl, furyloxy-C₁₋₆ alkyl, thienyl, thienyloxy, thienyl-C₁₋₆ alkyl or thienyloxy-C₁₋₆ alkyl, wherein said phenyl, furyl, thienyl or cycloalkyl are optionally substituted with 1-3 groups selected from C₁₋₆ alkyl, C₁₋₆ alkyloxy, C₁₋₆alkyloxy-C₁₋₆ alkyl, nitro, halogen, trihalomethyl, amino or hydroxy; or

(v) Furyl, furyloxy, thienyl, thienyloxy, naphthyl, naphthyloxy, phthalanyl or phthalanyloxy optionally substituted with 1-3 groups selected from C₁₋₆ alkyl, C₂₋₆ alkenyl, C₂₋₆ alkynyl, C₁₋₆ akyloxy, C₁₋₆ alkyloxy-C₁₋₆ alkyl, C₁₋₆ alkyloxy-C₁₋₆ alkyloxy, C₂₋₆ alkenyloxy-C₁₋₆ alkyl, C₁₋₆ alkyl substituted with 1-3 groups of hydroxy or halogen, C₁₋₆ alkylthio, C₁₋₆ alkylthio-C₁₋₆ alkyl, C₁₋₆ alkylthio-C₁₋₆ alkyloxy, C₁₋₆ alkenylthio-C₁₋₆ alkyl, C₁₋₆ alkylsulfonyl, halogen, trihalomethyl, cyano, nitro, amino, hydroxy, C₃₋₇ cycloalkyl, C₃₋₇ cycloalkyloxy, C₃₋₇ cycloalkyl-C₁₋₆ alkyl, C₃₋₇ cycloalkyloxy-C₁₋₆ alkyl, phenyl, phenyloxy, phenyl-C₁₋₆ alkyl, phenyl-C₂₋₆ alkenyl, phenyl-C₂₋₆ alkynyl, phenyloxy-C₁₋₆ alkyl, phenyloxy-C₂₋₆ alkenyl, phenyloxy-C₂₋₆ alkynyl, furyl, furyloxy, furyl-C₁₋₆ alkyl, furyloxy-C₁₋₆ alkyl, thienyl, thienyloxy, thienyl-C₁₋₆ alkyl and thienyloxy-C₁₋₆ alkyl, said phenyl, furyl, thienyl or cycloalkyl optionally substituted with 1-3 groups selected from C₁₋₆ alkyl, C₁₋₆ alkyloxy, C₁₋₆alkyloxy-C₁₋₆ alkyl, nitro, halogen, trihalomethyl, amino and hydroxy;

R^{5b} represents hydrogen or C 1-6 alkyl.

Preferred compounds of this invention are:

7-(2S-[3R-hydroxy-4-(3-phenoxy-phenyl)-butyl]-5-oxo-pyrrolidin-1-yl)-heptanoic acid;

7-(2S-[3R-hydroxy-4-(3-trifluoromethyl-phenyl)-butyl]-5-oxo-pyrrolidin-1-yl)-heptanoic acid;

7-(2S-[4-(3-chloro-phenyl)-3R-hydroxy-butyl]-5-oxo-pyrrolidin-1-yl)-heptanoic acid;

7-(2S-[3R-hydroxy-4-phenyl-butyl]-5-oxo-pyrrolidin-1-yl)-heptanoic acid;

7-(2R-[3S-hydroxy-4-phenyl-but-1-enyl]-5-oxo-pyrrolidin-1-yl)-heptanoic acid;

5S-(4-(3-chloro-phenyl)-3R-hydroxy-butyl)-1-(6-(2H-tetrazol-5-yl)-hexyl-pyrrolidin-2-one;

5S-(3R-hydroxy-4-(3-trifluoromethyl-phenyl)-butyl)-1-(6-(2H-tetrazol-5-yl)-hexyl-pyrrolidin-2-one;

5S-(3R-hydroxy-4-phenyl-butyl)-1-[6-(1H-tetrazol-5-yl)-hexyl]-pyrrolidin-2-one;

5R-(3S-hydroxy-4-phenyl-but-1-enyl)-1-[6-(1H-tetrazol-5-yl)-hexyl]-pyrrolidin-2-one;

7-{(2R)-2-[(1E)-3-hydroxy-4-methyl-4-phenylpent-1-enyl]-5-oxopyrrolidin-1-yl}heptanoic acid;

7-{(2R)-2-[(1E)-3-hydroxy-3-(1-phenylcyclopropyl)prop-1-enyl]-5-oxopyrrolidin-1-yl}heptanoic acid;

7-{(2R)-2-[(1E)-3-cyclohexyl-3-hydroxyprop-1-enyl]-5-oxopyrrolidin-1-yl}heptanoic acid;

11α,15α-dihydroxy-9-oxo-16-(3-methoxymethylphenyl)-17,18,19,20-tetranor-3,7-dithiaprost-13E-enoic acid or methyl ester thereof;

11α,15α-dihydroxy-9-oxo-16-(5-methoxymethylthiophen-2-yl)-17,18,19,20-tetranor-3,7-dithiaprost-13E-enoic acid or methyl ester thereof;

11α,15α-dihydroxy-9-oxo-16-phenyloxy-17,18,19,20-tetranor-3,7-dithiaprost-13E-enoic acid or methyl ester thereof;

11α,15α-dihydroxy-9-oxo-16-(4-methylphenyl)-17,18,19,20-tetranor-3,7-dithiaprost-13E-enoic acid or methyl ester thereof;

11α,15α-dihydroxy-9-oxo-16-(4-chlorophenyl)-17,18,19,20-tetranor-3,7-dithiaprost-13E-enoic acid or methyl ester thereof;

11α,15α-dihydroxy-9-oxo-16-(3-thienyl)-17,18,19,20-tetranor-3,7-dithiaprost-13E-enoic acid or methyl ester thereof;

11α,15α-dihydroxy-9-oxo-16-(2-naphthyl)-17,18,19,20-tetranor-3,7-dithiaprost-13E-enoic acid or methyl ester thereof;

11α,15α-dihydroxy-9-oxo-16-(5-phthalanyl)-17,18,19,20-tetranor-3,7-dithiaprost-13E-enoic acid or methyl ester thereof;

11α,15α-dihydroxy-9-oxo-16-(4-methoxyphenyl)-17,18,19,20-tetranor-3,7-dithiaprost-13E-enoic acid or methyl ester thereof;

11α,15α-dihydroxy-9-oxo-16-(4-methoxy-3-chlorophenyl)-17,18,19,20-tetranor-3,7-dithiaprost-13E-enoic acid;

11α,15α-dihydroxy-9-oxo-16-(3-trifluoromethylphenyl)-17,18,19,20-tetranor-3,7-dithiaprost-13E-enoic acid;

11α,15α-dihydroxy-9-oxo-16-phenyl-17,18,19,20-tetranor-3,7-dithiaprost-13E-enoic acid;

11α,15α-dihydroxy-9-oxo-17-phenyl-17,18,19,20-tetranor-3,7-dithiaprost-13E-enoic acid;

11 α,15α-dihydroxy-9-oxo-16α-methyl-16-phenyl-3,7-dithia-20-norprost-13E-enoic acid or methyl ester thereof;

11α,15α-dihydroxy-9-oxo-16-cyclohexyl-3,7-dithia-17,18,19,20-tetranorprost-13E-enoic acid;

11α,15α-dihydroxy-9-oxo-19,20-methano-3,7-dithiaprost-13E-enoic acid;

11α,15α-dihydroxy-9-oxo-16-cyclopentyll-3,7-dithia-17,18,19,20-tetranorprost-13E-enoic acid or methyl ester thereof;

11α,15α-dihydroxy-9-oxo-15-cyclohexyl-3,7-dithia-16,17,18,19,20-pentanorprost-13E-enoic acid or methyl ester thereof;

(11α,13E,15α)-9-oxo-11,15-dihydroxy-16-(3-methoxymethylphenyl)-17,18,19,20-tetranor-5-thiaprost-13-enoic acid, or methyl, n-propyl, i-propyl or n-butyl ester thereof;

(11α,13E,15α)-9-oxo-11,15-dihydroxy-16-(3-ethoxymethylphenyl)-17,18,19,20-tetranor-5-thiaprost-13-enoic acid, or methyl or ethyl ester thereof;

(11α,13E, 15α)-9-oxo-11,15-dihydroxy-16-(3-n-propyloxymethylphenyl)-17,18,19,20-tetranor-5-thiaprost-13-enoic acid, or methyl or t-butyl ester thereof;

(11α,15α)-9-oxo-11,15-dihydroxy-16-(3-methoxymethylphenyl)-17,18,19,20-tetranor-5-thiaprostanoic acid or methyl ester thereof;

(11α,15α,13E)-9-oxo-11,15-dihydroxy-16-methyl-16-(3-methoxymethylphenyl)-17,18,19,20-tetranor-5-thiaprost-13-enoic acid or methyl ester thereof;

(15α,13E)-9-oxo-15-dihydroxy-16-(3-methoxymethylphenyl)-17,18,19,20-tetranor-5-thiaprost-13-enoic acid or methyl ester thereof;

(11α,13E, 15α)-9-oxo-11,15-dihydroxy-16-(3-methyl-4-hydroxyphenyl)-17,18,19,20-tetranor-5-thiaprost-13-enoic acid or methyl ester thereof; or

(11α,15α,13E)-9-oxo-11,15-dihydroxy-16-(3-methyl-4-hydroxyphenyl)-17,18,19,20-tetranor-5-thiaprost-13-enoic acid or methyl ester thereof.

This is invention is also concerned with novel compounds which are useful as potent selective agonists of the EP₄ subtype of prostaglandin E2 receptors in the treatment of glaucoma and other conditions which are related to elevated intraocular pressure in the eye of a patient. Preferred compounds are:

7-{(2R)-2-[(1E)-3-hydroxy-3-(1-phenylcyclopropyl)prop-1-enyl]-5-oxopyrrolidin-1-yl}heptanoic acid;

7-{(2R)-2-[(1E)-3-cyclohexyl-3-hydroxyprop-1-enyl]-5-oxopyrrolidin-1-yl )heptanoic acid;

7-{(2R)-2-[(1E)-3-hydroxy-3-methyl-4-phenylbut-1-enyl]-5-oxopyrrolidin-1-yl}heptanoic acid or its pharmaceutically acceptable salt or ester thereof, and

3S-(3-hydroxy-4-phenylbutyl)-2R-[6-(1H-tetrazol-5-yl)-hexyl]cyclopentanone.

The invention is described herein in detail using the terms defined below unless otherwise specified.

The term “alkyl” refers to a monovalent alkane (hydrocarbon) derived radical containing from 1 to 10 carbon atoms unless otherwise defined. It may be straight or branched. Preferred alkyl groups include methyl, ethyl, propyl, isopropyl, butyl and t-butyl. When the alkyl group is said to be substituted with an alkyl group, this is used interchangeably with “branched alkyl group”.

Cycloalkyl is a species of alkyl containing from 3 to 15 carbon atoms, without alternating or resonating double bonds between carbon atoms. It may contain from 1 to 4 rings which are fused. Examples of cycloalkyl groups include cyclopropyl, cyclobutyl, cyclopentyl, cyclohexyl, and cycloheptyl.

Alkoxy refers to C₁-C₆ alkyl-O—, with the alkyl group optionally substituted as described herein. Examples of alkoxy groups include methoxy, ethoxy, propoxy, butoxy and isomeric groups thereof.

Alkenyl refers to alkyl groups having a double bond, including vinyl, propenyl, butenyl, pentenyl, hexenyl, heptenyl, octenyl and isomeric groups thereof.

Alkynyl refers to alkyl groups have a triple bond, including ethynyl, propynyl, butynyl, pentynyl, hexynyl, heptynyl, octynyl and isomers thereof.

Halogen (halo) refers to chlorine, fluorine, iodine or bromine.

The term “agonist” as used herein means that the EP₄ subtype compounds of formula Ia, Ib, Ic, Id, Ie or If interact with the EP₄ receptor to produce maximal, super maximal or submaximal effects compared to the natural agonist, PGE₂. See Goodman and Gilman, The Pharmacological Basis of Therapeutics, 9^{th} edition, 1996, chapter 2.

This invention is also concerned with a method of treating ocular hypertension or glaucoma by administering to a patient in need thereof one of the compounds of formula Ia, Ib, Ic, Id, Ie or If alone or in combination with: a β-adrenergic blocking agent, such as timolol, betaxolol, levobetaxolol, carteolol, levobunolol; a parasympathomimetic agent, such as pilocarpine; a sympathomimetic agent, such as epinephrine, iopidine, brimonidine, clonidine, para-aminoclonidine; a carbonic anhydrase inhibitor, such as dorzolamide, acetazolamide, metazolamide or brinzolamide; a prostaglandin such as latanoprost, travaprost, unoprostone, rescula, S1033 (compounds set forth in U.S. Pat. Nos. 5,889,052; 5,296,504; 5,422,368; and 5,151,444); a hypotensive lipid such as lumigan and the compounds set forth in U.S. Pat. No. 5,352,708; a neuroprotectant disclosed in U.S. Pat. No. 4,690,931, particularly eliprodil and R-eliprodil as set forth in WO 94/13275, including memantine; or an agonist of 5-HT2 receptors as set forth in PCT/US00/31247, particularly 1-(2-aminopropyl)-3-methyl-1H-imdazol-6-ol fumarate and 2-(3-chloro-6-methoxy-indazol-1-yl)-1-methyl-ethylamine.

This invention is further concerned with a process for making a pharmaceutical composition comprising a compound of formula Ia, Ib, Ic, Id, Ie or If and a pharmaceutically acceptable carrier.

The claimed compounds bind strongly and act on PGE₂ receptor, particularly on the EP₄ subtype receptor and therefore are useful for preventing and/or treating glaucoma and/or ocular hypertension. Use of the compounds of formula Ia, Ib, Ic, Id, Ie or If for the manufacture of a medicament for treating glaucoma and/or ocular hypertension is also included in this invention.

Dry eye is a common ocular surface disease afflicting millions of people. Although it appears that dry eye may result from a number of unrelated pathogenic causes, the common end result is the breakdown of the tear film, which results in dehydration of the exposed outer surface of the eye. (Lemp, Report of the Nation Eye Institute/Industry Workshop on Clinical Trials in Dry Eyes, The CLAO Journel, 21(4):221-231 (1995)). One cause for dry eye is the decreased mucin production by the conjunctival cells and/or corneal epithelial cells of mucin, which protects and lubricates the ocular surface (Gipson and Inatomi, Mucin genes expressed by ocular surface epithelium. Progress in Retinal and Eye Research, 16:81-98 (1997)). Functional EP4 receptors have been found in human conjuctival epithelial cells (see U.S. Pat. No. 6,344,477, incorporated by reference in its entirety) and it is appreciated that both human corneal epithelial cells (Progress in Retinal and Eye Research, 16:81-98(1997)) and conjuctival cells (Dartt et al. Localization of nerves adjacent to goblet cells in rat conjucntiva. Current Eye Research, 14:993-1000 (1995)) are capable of secreting mucins. Thus, the compounds of formula Ia, Ib, Ic, Id, Ie or If are useful for treating dry eye. Use of the compounds of formula Ia, Ib, Ic, Id, Ie or If for the manufacture of a medicament for treating dry eye is also included in this invention.

Macular edema is swelling within the retina within the critically important central visual zone at the posterior pole of the eye. An accumulation of fluid within the retina tends to detach the neural elements from one another and from their local blood supply, creating a dormancy of visual function in the area. It is believed that EP₄ agonist which lower IOP are useful for treating diseases of the macular such as macular edema or macular degeneration. Thus, another aspect of this invention is a method for treating macular edema or macular degeneration.

Glaucoma is characterized by progressive atrophy of the optic nerve and is frequently associated with elevated intraocular pressure (IOP). It is possible to treat glaucoma, however, without necessarily affecting IOP by using drugs that impart a neuroprotective effect. See Arch. Ophthalmol. Vol. 112, January 1994, pp. 37-44; Investigative Ophthamol. & Visual Science, 32, 5, April 1991, pp. 1593-99. It is believed that EP₄ agonist which lower IOP are useful for providing a neuroprotective effect. They are also believed to be effective for increasing retinal and optic nerve head blood velocity and increasing retinal and optic nerve oxygen by lowering IOP, which when coupled together benefits optic nerve health. As a result, this invention further relates to a method for increasing retinal and optic nerve head blood velocity, or increasing retinal and optic nerve oxygen tension or providing a neuroprotective effect or a combination thereof by using an EP₄ agonist of formula Ia, Ib, Ic, Id, Ie or If. Use of the compounds of formula Ia, Ib, Ic, Id, Ie or If for the manufacture of a medicament for increasing retinal and optic nerve head blood velocity, or increasing retinal and optic nerve oxygen tension or providing a neuroprotective effect or a combination thereof is also included in this invention.

The compounds produced in the present invention are readily combined with suitable and known pharmaceutically acceptable excipients to produce compositions which may be administered to mammals, including humans, to achieve effective IOP lowering. Thus, this invention is also concerned with a method of treating ocular hypertension or glaucoma by administering to a patient in need thereof one of the compounds of formula Ia, Ib, Ic, Id, Ie or If alone or in combination with: a β-adrenergic blocking agent, such as timolol, betaxolol, levobetaxolol, carteolol or levobunolol; a parasympathomimetic agent such as pilocarpine; a sympathomimetic agent such as, epinephrine, iopidine, brimonidine, clonidine, para-aminoclonidine; a carbonic anhydrase inhibitor, such as, dorzolamide, acetazolamide, metazolamide or brinzolamide; a prostaglandin such as latanoprost, travaprost, unoprostone, rescula, S1033 (compounds set forth in U.S. Pat. Nos. 5,889,052; 5,296,504; 5,422,368; and 5,151,444); a hypotensive lipid such as lumigan and the compounds set forth in U.S. Pat. No. 5,352,708; a neuroprotectant disclosed in U.S. Pat. No. 4,690,931, particularly eliprodil and R-eliprodil as set forth in WO 94/13275, including memantine; or an agonist of 5-HT2 receptors as set forth in PCT/US00/31247, particularly 1-(2-aminopropyl)-3-methyl-1H-imdazol-6-ol fumarate and 2-(3-chloro-6-methoxy-indazol-1-yl)-1-methyl-ethylamine.

Thus, this invention is also concerned with a method for increasing retinal and optic nerve head blood velocity, or increasing retinal and optic nerve oxygen tension or providing a neuroprotective effect or a combination thereof by administering to a patient in need thereof one of the compounds of formula Ia, Ib, Ic, Id, Ie or If alone or in combination with: a β-adrenergic blocking agent, such as timolol, betaxolol, levobetaxolol, carteolol or levobunolol; a parasympathomimetic agent such as pilocarpine; a sympathomimetic agent such as, epinephrine, iopidine, brimonidine, clonidine, para-aminoclonidine; a carbonic anhydrase inhibitor, such as, dorzolamide, acetazolamide, metazolamide or brinzolamide; a prostaglandin such as latanoprost, travaprost, unoprostone, rescula, S1033 (compounds set forth in U.S. Pat. Nos. 5,889,052; 5,296,504; 5,422,368; and 5,151,444); a hypotensive lipid such as lumigan and the compounds set forth in U.S. Pat. No. 5,352,708; a neuroprotectant disclosed in U.S. Pat. No. 4,690,931, particularly eliprodil and R-eliprodil as set forth in WO 94/13275, including memantine; or an agonist of 5-HT2 receptors as set forth in PCT/US00/31247, particularly 1-(2-aminopropyl)-3-methyl-1H-imdazol-6-ol fumarate and 2-(3-chloro-6-methoxy-indazol-1-yl)-1-methyl-ethylamine.

This invention is further concerned with a method for treating macular edema or macular degeneration by administering to a patient in need thereof one of the compounds of formula Ia, Ib, Ic, Id, Ie or If alone or in combination with: a β-adrenergic blocking agent, such as timolol, betaxolol, levobetaxolol, carteolol, levobunolol; a parasympathomimetic agent, such as pilocarpine; a sympathomimetic agent, such as epinephrine, iopidine, brimonidine, clonidine, para-aminoclonidine; a carbonic anhydrase inhibitor, such as dorzolamide, acetazolamide, metazolamide or brinzolamide; a prostaglandin such as latanoprost, travaprost, unoprostone, rescula, S1033 (compounds set forth in U.S. Pat. Nos. 5,889,052; 5,296,504; 5,422,368; and 5,151,444); a hypotensive lipid such as lumigan and the compounds set forth in U.S. Pat. No. 5,352,708; a neuroprotectant disclosed in U.S. Pat. No. 4,690,931, particularly eliprodil and R-eliprodil as set forth in WO 94/13275, including memantine; or an agonist of 5-HT2 receptors as set forth in PCT/US00/31247, particularly 1-(2-aminopropyl)-3-methyl-1H-imdazol-6-ol fumarate and 2-(3-chloro-6-methoxy-indazol-1-yl)-1-methyl-ethylamine.

The herein examples illustrate but do not limit the claimed invention. Each of the claimed compounds are EP₄ agonists and are useful for a number of physiological ocular disorders. The compounds produced in the present invention are readily combined with suitable and known pharmaceutically acceptable excipients to produce compositions which may be administered to mammals, including humans, to achieve effective IOP lowering.

The EP₄ agonist used in the instant invention can be administered in a therapeutically effective amount intravaneously, subcutaneously, topically, transdermally, parenterally or any other method known to those skilled in the art. Ophthalmic pharmaceutical compositions are preferably adapted for topical administration to the eye in the form of solutions, suspensions, ointments, creams or as a solid insert. Ophthalmic formulations of this compound may contain from 0.001 to 5% and especially 0.001 to 0.1% of medicament. Higher dosages as, for example, up to about 10% or lower dosages can be employed provided the dose is effective in reducing intraocular pressure, treating glaucoma, increasing blood flow velocity or oxygen tension. For a single dose, from between 0.001 to 5.0 mg, preferably 0.005 to 2.0 mg, and especially 0.005 to 1.0 mg of the compound can be applied to the human eye.

The pharmaceutical preparation which contains the compound may be conveniently admixed with a non-toxic pharmaceutical organic carrier, or with a non-toxic pharmaceutical inorganic carrier. Typical of pharmaceutically acceptable carriers are, for example, water, mixtures of water and water-miscible solvents such as lower alkanols or aralkanols, vegetable oils, peanut oil, polyalkylene glycols, petroleum based jelly, ethyl cellulose, ethyl oleate, carboxymethyl-cellulose, polyvinylpyrrolidone, isopropyl myristate and other conventionally employed acceptable carriers. The pharmaceutical preparation may also contain non-toxic auxiliary substances such as emulsifying, preserving, wetting agents, bodying agents and the like, as for example, polyethylene glycols 200, 300, 400 and 600, carbowaxes 1,000, 1,500, 4,000, 6,000 and 10,000, antibacterial components such as quaternary ammonium compounds, phenylmercuric salts known to have cold sterilizing properties and which are non-injurious in use, thimerosal, methyl and propyl paraben, benzyl alcohol, phenyl ethanol, buffering ingredients such as sodium borate, sodium acetates, gluconate buffers, and other conventional ingredients such as sorbitan monolaurate, triethanolamine, oleate, polyoxyethylene sorbitan monopalmitylate, dioctyl sodium sulfosuccinate, monothioglycerol, thiosorbitol, ethylenediamine tetracetic acid, and the like. Additionally, suitable ophthalmic vehicles can be used as carrier media for the present purpose including conventional phosphate buffer vehicle systems, isotonic boric acid vehicles, isotonic sodium chloride vehicles, isotonic sodium borate vehicles and the like. The pharmaceutical preparation may also be in the form of a microparticle formulation. The pharmaceutical preparation may also be in the form of a solid insert. For example, one may use a solid water soluble polymer as the carrier for the medicament. The polymer used to form the insert may be any water soluble non-toxic polymer, for example, cellulose derivatives such as methylcellulose, sodium carboxymethyl cellulose, (hydroxyloweralkyl cellulose), hydroxyethyl cellulose, hydroxypropyl cellulose, hydroxypropylmethyl cellulose; acrylates such as polyacrylic acid salts, ethylacrylates, polyactylamides; natural products such as gelatin, alginates, pectins, tragacanth, karaya, chondrus, agar, acacia; the starch derivatives such as starch acetate, hydroxymethyl starch ethers, hydroxypropyl starch, as well as other synthetic derivatives such as polyvinyl alcohol, polyvinyl pyrrolidone, polyvinyl methyl ether, polyethylene oxide, neutralized carbopol and xanthan gum, gellan gum, and mixtures of said polymer.

Suitable subjects for the administration of the formulation of the present invention include primates, man and other animals, particularly man and domesticated animals such as cats, rabbits and dogs.

The pharmaceutical preparation may contain non-toxic auxiliary substances such as antibacterial components which are non-injurious in use, for example, thimerosal, benzalkonium chloride, methyl and propyl paraben, benzyldodecinium bromide, benzyl alcohol, or phenylethanol; buffering ingredients such as sodium chloride, sodium borate, sodium acetate, sodium citrate, or gluconate buffers; and other conventional ingredients such as sorbitan monolaurate, triethanolamine, polyoxyethylene sorbitan monopalmitylate, ethylenediamine tetraacetic acid, and the like.

The ophthalmic solution or suspension may be administered as often as necessary to maintain an acceptable IOP level in the eye. It is contemplated that administration to the mammalian eye will be from once up to three times daily.

For topical ocular administration the novel formulations of this invention may take the form of solutions, gels, ointments, suspensions or solid inserts, formulated so that a unit dosage comprises a therapeutically effective amount of the active component or some multiple thereof in the case of a combination therapy.

The formula Ia, Ib, Ic, Id and Ie agonists generally have an EC₅₀ value from about 0.001 nM to about 100 microM, although agonists with activities outside this range can be useful depending upon the dosage and route of administration. In a subclass of the present invention, the agonists have an EC₅₀ value of from about 0.01 microM to about 10 microM. In a further subclass of the present invention, the agonists have an EC₅₀ value of from about 0.1 microM to about 10 microM. EC₅₀ is a common measure of agonist activity well known to those of ordinary skill in the art and is defined as the concentration or dose of an agonist that is needed to produce half, i.e. 50%, of the maximal effect. See also, Goodman and Gilman's, The Pharmacologic Basis of Therapeutics, 9th edition, 1996, chapter 2, E. M. Ross, Pharmacodynamics, Mechanisms of Drug Action and the Relationship Between Drug Concentration and Effect,

The compounds of this invention and methods for their preparation are disclosed in U.S. Pat. No. 6,043,275, European patent Nos. EP0855389 and EP 1097922, PCT publications WO 01/46140, WO 01/66518, WO 01/49661 and WO 00/03980, U.S. patent application Publication U.S. 2002/0065308 (which describes compounds of formula Ie and methods for preparing these compounds) and WO 9731640 (which described compound If) the contents of which are all incorporated by reference in their entirety, said compounds can be made, with some modification, in accordance with the same references. The following non-limiting examples, given by way of illustration, are demonstrative of the present invention.

Medium pressure chromatography was performed using a Biotage purification 5 system (Biotage, Dyax Corporation, Charlottesville, Va.) under nitrogen pressure. Flash chromatography was performed with either Baker Silica Gel (40 Elm) (J. T. Baker, Phillipsburg, N.J.) or Silica Gel 60 (EM Sciences, Gibbstown, N.J.) in glass columns under low nitrogen pressure. Radial Chromatography was performed using a Chromatotron (model 7924T, Harrison Research). Preparative Chromatography was performed using Analtech Uniplates Silica Gel GF (20×2 cm) (Analtech, Inc. Newark, Del.). Dimethylformamide (DMF), tetrahydrofuran (THF), and dichloromethane (CH₂Cl₂) used as reaction solvents were the anhydrous grade supplied by Aldrich Chemical Company (Milwaukee, Wis.). The term ‘concentrated’ refers to removal of solvent at water aspirator pressure on a rotary evaporator. The abbreviation h stands for hours. The term “TBAF” refers to tetrabutylammonium fluoride. The term “DMAP” refers to dimethylaminopyridine. The terms “dichloromethane” and “methylene chloride” are synonymous and are used interchangeably throughout this specification and in the Examples and Preparations.

### EXAMPLE 1

### 7-(2S-[4-(3-Chloro-phenyl)-3R-hydroxy-butyl]-5-oxo-pyrrolidin-1-yl)-heptanoic Acid

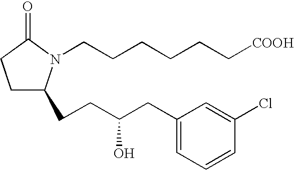

Step A:

7{2R-[4-(3-Chloro-phenyl)-3-oxo-but-1-enyl]-5-oxo-pyrrolidin-1-yl-heptanoic Acid Ethyl Ester.

To a solution of [3-(3-chloro-phenyl)-2-oxo-propyl]-phosphonic acid dimethyl ester (2.66 g, 9.63 mmol) in THF (35 mL) at O° C. was added NaH (60% by weight in oil, 426 mg, 10.7 mmol) portionwise. The reaction mixture was stirred at room temperature for 40 minutes. The reaction mixture was cooled to O° C. and a solution of 7-(2R-formyl-5-oxo-pyrrolidin-1-yl)-heptanoic acid ethyl ester (assumed 10.6 mmol, prepared according to the method described in Preparation 7, but using different amounts of reagents) in THF was added and the reaction was stirred for 18 hours. AcOH was added and the reaction mixture was diluted with EtOAc. The organic solution was washed consecutively with saturated NaHCO₃ solution (2×), water (1×), and brine (1×). The organic solution was dried (MgSO₄), filtered and concentrated. The residue was purified by medium pressure chromatography eluting with 15% acetone in toluene to provide 7-{2R-[4-(3-chloro-phenyl)-3-oxo-but-1-enyl]-5-oxopyrrolidin-1-yl}-heptanoic acid ethyl ester. ¹H NMR (CDCl₃) δ 7.27-7.15 (m, 3H), 7.08 (m, 1H), 6.66 (dd, 1H), 6.20 (d, 1H), 4.17 (m, 1H), 4.11 (q, 2H), 3.82 (s, 2H), 3.55 (m, 1H), 2.72 (m, 1H), 2.46-2.23 (m, 5H), 1.79 (m, 1 H), 1.58 (m, 2H), 1.47-1.20 (m, 9H); MS 420.2 (M+1), 418.2 (M−1).

Step B:

7-{2R-[4-(3-Chloro-phenyl)-3S-hydroxy-but-1-enyl]-5-oxo-pyrrolidin-1-yl}heptanoic Acid Ethyl Ester

To a solution of 7-{2R -[4-(3-chloro-phenyl)-3-oxo-but-1-enyl]-5-oxo-pyrrolidin-1-yl)-heptanoic acid ethyl ester (2.1 g, 5.0 mmol) in anhydrous CH₂Cl₂ (200 mL) was added (R)-2-methyl-CBS-oxazaborolidine (1 M in toluene, 5 mL, 5 mmol) and the solution was cooled to −45° C. The reaction mixture was stirred for 20 minutes and catecholborane (1 M in THE, 15 mL, 15 mmol) was added. The reaction mixture was stirred for 18 h at −45° C. Aqueous HCl (1 N, 100 mL) was added and the reaction mixture was stirred at room temperature for 18 h. The acidic aqueous layer was separated and the organic solution was washed with ice-cold 1N NaOH (2×) followed by brine (1×). The organic solution was dried (MgSO₄), filtered and concentrated. Purification by medium pressure chromatography (1:1 EtOAc:hexanes to 80% EtOAc in hexanes) provided 7-{2R-[4-(3-chloro-phenyl)-3S-hydroxy-but-1-enyl]-5-oxo-pyrrolidin-1-yl)-heptanoic acid ethyl ester as an approximate 6:1 mixture of 3S:3R alcohol diastereomers by ¹H NMR. ¹H NMR (CDCl₃) δ 7.23-7.17 (m, 3H), 7.06 (m, 1H), 5.67 (dd, 1H), 5.46 (dd, 1H), 4.37 (m, 1H), 4.08 (q, 2H), 4.00 (m, 1H), 3.44 (m, 1H), 2.80 (m, 2H), 2.67 (m, 1H), 2.41-2.12 (m, 5H), 1.70-1.20 (m, 13H); MS 422.3 (M+1).

Step C:

7-{2S-[4-(3-Chloro-phenyl)-3R-hydroxy-butyl]-5-oxo-pyrrolidin-1-yl}-heptanoic Acid Ethyl Ester.

To a solution of 7-{2R-[4-(3-chloro-phenyl)-3S-hydroxy-but-1-enyl]-5-oxo-pyrrolidin-1-yl)-heptanoic acid ethyl ester (800 mg, 1.90 mmol) in EtOH (50 mL) was added 10% palladium on carbon (80 mg). The reaction mixture was hydrogenated on a Parr shaker at 45 psi for 4.5 hours. The catalyst was removed via filtration through Celite® with the aid of EtOH. Purification by medium pressure chromatography (1:1 hexanes:EtOAc to 4:1 EtOAc:hexanes) provided 7-{2S-[4-(3-chloro-phenyl)-3R-hydroxy-butyl]-5-oxo-pyrrolidin-1-yl}-heptanoic acid ethyl ester. ¹H NMR (CDCl₃) δ 7.27-7.21 (m, 3H), 7.09 (m, 1H), 4.10 (m, 2H), 3.84 (m, 1H), 3.61 (m, 2H), 2.90 (m, 1H), 2.78 (dd, 1H), 2.68 (m, 1H), 2.47-2.25 (m, 4H), 2.12 (m, 1H), 1.92-1.22 (m, 17H); MS 424.3 (M+1).

Step D:

7-{2S-[4-(3-Chloro-phenyl)-3R-hydroxy-butyl]-5-oxo-pyrrolidin-1-yl}-heptanoic Acid

To a solution of 7-{2S-[4-(3-chloro-phenyl)-3R-hydroxy-butyl]-5-oxo-pyrrolidin-1-yl}-heptanoic acid ethyl ester (595 mg, 1.40 mmol) in EtOH (25 mL) was added 2N NaOH (6 mL). The reaction was stirred for 24 h at room temperature and was concentrated in vacuo to about ¾ the original volume. Aqueous 1N HCl was added to obtain a pH of about 2. The aqueous solution was washed with methylene chloride (3×). The combined organic layers were washed with water followed by brine. The organic solution was dried (MgSO₄), filtered and concentrated to provide the title compound of Example 1. ¹H NMR (CDCl₃) δ 7.26-7.18 (m, 3H), 7.08 (m, 1H), 3.84 (m, 1H), 3.58 (m, 21-1), 2.90 (m, 1H), 2.77 (dd, 1H), 2.68 (m, 1H), 2.43-2.28 (m, 4H), 2.10 (m, 1H), 1.78 (m, 1H), 1.66-1.22 (m, 13H); MS 396.2 (M+1), 394.2 (M−1).

### EXAMPLE 2

### 7-{2S-[3R-Hydroxy-4-(3-trifluoromethyl-phenyl)-butyl]-5-oxo-pyrrolidin-1-yl}-heptanoic Acid

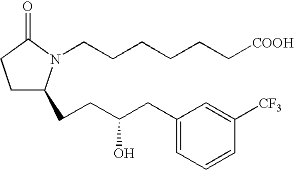

Step A:

7-{2-Oxo-5R-[3-oxo-4-(3-trifluoromethyl-phenyl)-but-1-enyl]-pyrrolidin-1-yl}-heptanoic Acid Ethyl Ester.

Following the procedure described for Example 1, Step A, the anion derived from [2-oxo-3-(3-trifluoromethyl-phenyl)-propyl]-phosphonic acid dimethyl ester (4.16 g, 13.40 mmol) and NaH (60% in oil, 590 mg, 14.7 mmol) was reacted with 7-(2R-formyl-5-oxo-pyrrolidin-1-yl)-heptanoic acid ethyl ester (assumed 14.74 mmol) over 24 h. Purification by medium pressure chromatography (solvent gradient 20% EtOAc in hexanes to EtOAc) provided 7-{2-oxo-5R-[3-oxo-4-(3trifluoromethyl-phenyl)-but-1-enyl]-pyrrolidin-1-yl)-heptanoic acid ethyl ester. ¹H NMR (CDCl₃) δ 7.52 (d, 1H), 7.44 (m, 2H), 7.37 (d, 1H), 6.67 (dd, 1H), 6.22 (d, 1H), 4.80 (m, 1H), 4.08 (q, 2H), 3.90 (s, 2H), 3.54 (m, 1H), 2.70 (m, 1H), 2.37 (m, 2H), 2.24 (m, 3H), 1.78 (m, 1H), 1.56 (m, 2H), 1.44-1.20 (m, 9H); MS 454.2 (M+1), 452.2 (M−1).

Step B:

7-{2R-[3S-Hydroxy-4-(3-trifluoromethyl-phenyl)-but-1-enyl]-5-oxo-pyrrolidin-1-yl}-heptanoic Acid Ethyl Ester.

To a solution of 7-{2-oxo-5R-[3-oxo-4-(3-trifluoromethyl-phenyl)-but-1-enyl)-pyrrolidin-1-yl}-heptanoic acid ethyl ester (1.5 g, 3.31 mmol) and (R)-2-methyl-CBS-oxazaborolidine (1M in toluene, 0.5 mL, 0.5 mmol) in CH₂Cl₂ (100 mL) at −45° C. was added catecholborane (1 M in THF, 9.9 mL, 9.9 mmol) dropwise. The solution was stirred for 24 h at −45° C. and 1N HCl was added. The reaction mixture was stirred at room temperature for 1 h and the layers were separated. The aqueous solution was washed with CH₂Cl₂ (2×) and the combined organic layers were washed consecutively with ice-cold 0.5N NaOH and brine (two times). The organic solution was dried (MgSO₄), filtered and concentrated. Purification by medium pressure chromatography (50% EtOAc in hexanes to 60% EtOAc in hexanes to 80% EtOAc in hexanes to EtOAc to 5% MeOH in CH₂Cl₂) provided 7-{2R-[3S-hydroxy-4 (3-trifluoromethyl-phenyl)-but-1-enyl]-5-oxo-pyrrolidin-1-yl}-heptanoic acid ethyl ester as an approximate 5.5:1 mixture of 3S:3R alcohol diastereomers by HPLC analysis. ¹HMR(CDCl₃)δ 7.51-7.35 (m, 4H), 5.72 (dd, 1H), 5.50 (dd, 1H), 4.44 (m, 1H), 4.09 (q, 2H), 4.01 (m, 1H), 3.44 (m, 1H), 2.90 (d, 2H), 2.71 (m, 1H), 2.37-2.12 (m, 5H), 1.70-1.21 (m, 13H); MS 456.3 (M+1), 454.3 (M−1).

Step C:

7-{2S-[3R-Hydroxy-4-(3-trifluoromethyl-phenyl)-butyl]-5-oxo-pyrrolidin-1-yl}-heptanoic Acid Ethyl Ester.

Following the procedure described for Example 1, Step C, a solution of 7-{2R-[3S-hydroxy-4-(3-trifluoromethyl-phenyl)-but-1-enyl]-5-oxo-25 pyrrolidin-1-yl}-heptanoic acid ethyl ester (1.18 g, 2.59 mmol) in EtOH (50 mL was hydrogenated in the presence of 10% palladium on carbon (120 mg) at 40-45 psi on a Parr shaker for 24 h. Purification by medium pressure chromatography (50% EtOAc in hexanes to EtOAc to 1% MeOH in CH₂Cl₂ to 3% MeOH in CH₂Cl₂ to 5% MeOH in CH₂Cl₂ to 10% MeOH in CH₂Cl₂) provided 7-{2S-[3R-hydroxy-4-(3 trifluoromethyl-phenyl)-butyl]-5-oxo-pyrrolidin-1-yl}-heptanoic acid ethyl ester. ¹H NMR (CDCl₃) δ 7.51-7.39 (m, 4H), 4.09 (q, 2H), 3.86 (m, 1H), 3.60 (m, 2H), 2.89 (m, 2H), 2.76 (m, 1H), 2.33 (m, 4H), 2.11 (m, 1H), 1.80 (m, 1H), 1.68-1.21 (m, 16H).

Step D:

7-{2S-[3R-Hydroxy-4-(3-trifluoromethyl-phenyl)-butyl]-5-oxo-pyrrolidin-1-yl}-heptanoic Acid

Following the procedure described for Example 1, Step D, 7-{2S-[3R-hydroxy-4-(3-trifluoromethyl-phenyl)-butyl]-5-oxo-pyrrolidin-1-yl}-heptanoic acid ethyl ester (1.93 g, 4.22 mmol) was hydrolyzed with 6N NaOH (26 mL) in EtOH (52 mL) over 24 h. Purification by medium pressure chromatography (EtOAc to 1% MeOH in CH₂Cl₂ to 3% MeOH in CH₂Cl₂ to 5% MeOH in CH₂Cl₂to 10% MeOH in CH₂Cl₂ provided 7-{2S-[3R-hydroxy-4-(3-trifluoromethyl-phenyl)-butyl]-5-oxo-pyrrolidin-1-yl}heptanoic acid. ¹H NMR (CDCl₃) δ 7.51-7.39 (m, 4H), 3.88 (m, 1H), 3.58 (m, 2H), 2.84 (m, 3H), 2.34 (m, 4H), 2.10 (m, 1H), 1.80 (m, 1H), 1.67-1.26 (m, 13H); MS 430.4 (M+1).

Step E:

Sodium Salt of 7-{2S-[3R-Hydroxy-4-(3-trifluoromethyl-phenyl)-butyl]-5-oxo-Pyrrolidin-1-yl}-heptanoic Acid

To a solution of 7-{2S-[3R-hydroxy-4-(3-trifluoromethyl-phenyl)-butyl]-5-oxo-pyrrolidin-1-yl}-heptanoic acid (1.66 g, 3.87 mmol) in EtOH (116 mL) was added NaHCO₃ (325 mg, 3.87 mmol) in water (3 mL). The reaction mixture was stirred for 5 h and was concentrated in vacuo. The residue was azeotroped with CH₂Cl₂ (3×) to provide the sodium salt of the title compound of Example 2. ¹H NMR (CD₃OD) δ 7.48 (m, 4H), 3.80 (m, 1H), 3.69 (m, 1H), 3.52 (m, 1 H), 2.94 (m, 1H), 2.81 (m, 2H), 2.32 (m, 2H), 2.13 (m, 3H), 1.81 (m, 1H), 1.69-1.26 (m, 13H); MS 430.3 (M-Na+1), 428.2 (M−Na−1).

### EXAMPLE 3

### 5S-[4-(3-Chloro-phenyl)-3-hydroxy-butyl]-1-[6-(2H-tetrazol-5-yl)-hexyl]-pyrrolidin-2-one

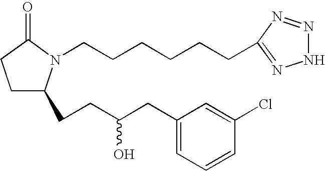

Step A:

7-{2R-[4-(3-Chloro-phenyl)-3-oxo-but-1-enyl]-5-oxo-pyrrolidin-1-yl}-heptanenitrile

Following the procedure described for Example 1, Step A, the anion derived from [3-(3-chloro-phenyl)-2-oxo-propyl]-phosphonic acid dimethyl ester (3.35 g, 12.12 mmol) and NaH (60% in oil, 533 mg, 13.3 mmol) was reacted with 7-(2R-formyl-5-oxo-pyrrolidin-1-yl)-heptanenitrile (assumed 13.3 mmol) over 18 h. Purification by medium pressure chromatography (20% EtOAc in hexanes to 80% EtOAc in hexanes) provided 7-{2R-[4-(3-chloro-phenyl)-3-oxo-but-1-enyl]-5-oxo pyrrolidin-1-yl}-heptanenitrile. ¹H NMR (CDCl₃) δ 7.24 (m, 2H), 7.17 (s, 1 H), 7.06 (m, 1 H), 6.64 (dd, 1 H), 6.20 (d, 1 H), 4.15 (m, 1 H), 3.80 (s, 2H), 3.50 (m, 1 H), 2.72 (m, 1 H), 2.46-2.20 (m, 5H), 1.78 (m, 1 H), 1.59 (m, 2H), 1.40 (m, 4H), 51.24 (m, 2H).

Step B:

7-{2S-[4-(3-Chloro-phenyl)-3-oxo-butyl-5-oxo-pyrrolidin-1-yl}heptanenitrile

Following the procedure described for Example 1, Step C, 7-{2R-[4(3-chloro-phenyl)-3-oxo-but-1-enyl]-5-oxo-pyrrolidin-1-yl}-heptanenitrile (860 mg, 2.31 mmol) in MeOH (40 mL) was hydrogenated in the presence of 10% palladium on carbon (86 mg) for 1 h. Purification by radial chromatography (hexanes; to 20% EtOAc in hexanes to 70% EtOAc in hexanes) provided 7-{2S-[4-(3-chloro-phenyl) 3-oxo-butyl]-5-oxo-pyrrolidin-1-yl}-heptanenitrile.

Step C:

7-{2S-[4-(3-Chloro-phenyl)-3-hydroxy-butyl]-5-oxo-pyrrolidin-1-yl]heptanenitrile

To a solution of 7-{2S-[4-(3-chloro-phenyl)-3-oxo-butyl]-5-oxo-15 pyrrolidin-1-yl}-heptanenitrile (730 mg, 1.87 mmol) in MeOH (30 mL) at 0° C. was added NaBH₄ (35 mg, 0.921 mmol). The reaction mixture was stirred at 0° C. for 45 minutes and water was added. The volatiles were removed in vacuo and the remaining aqueous solution was diluted with methylene chloride. The organic solution was washed with water followed by brine, dried (MgSO₄), filtered and concentrated. Purification by medium pressure chromatography (1:1 hexanes:EtOAc to EtOAc to 3% MeOH in CH₂Cl₂) provided 7-{2S-[4-(3-chlorophenyl)-3-hydroxy-butyl]-5-oxo-pyrrolidin-1-yl)-heptanenitrile. ¹H NMR (CDCl₃) δ 7.22 (m, 3H), 7.07 (d, 1H), 3.80 (m, 1 H), 3.57 (m, 2H), 2.88 (m, 1 H), 2.78 (m, 1 H), 2.64 (m, 1 H), 2.31 (m, 4H), 2.10 (m, 1 H), 1.65-1.22 (m, 141-1): MS 377.3 (M+I).

Step D:

5S-[4-(3-Chloro-phenyl)-3-hydroxy-butyl]-1-[6-(2H-tetrazol-5-yl)-hexyl]-Pyrrolidin-2-one

A solution of 7-{2S-[4-(3-chloro-phenyl)-3-hydroxy-butyl]-5-oxo-pyrrolidin-1-yl-heptanenitrile (730 mg, 1.94 mmol), trimethylsilylazide (0.63 mL, 0.475 mmol) and dibutyltin oxide (96 mg, 3.87 mmol) in toluene (30 mL) was heated at reflux for 18 h. The volatiles were removed in vacuo and the residue was diluted with CH₂Cl₂. The organic solution was washed with 1 N HCl followed by brine. The organic solution was dried (MgSO₄), filtered, and concentrated. The residue was purified by medium pressure chromatography eluting with EtOAc to 2% MeOH in CH₂Cl₂ to 7% MeOH in CH₂Cl₂ to give the TMS complex. The residue was diluted with MeOH and 2N HCl was added and the solution was stirred for 40 minutes. The solution was diluted with CH₂Cl₂ and the organic layer was washed with water followed by brine.

The organic solution was dried (MgSO₄), filtered, and concentrated. The residue was purified by medium pressure chromatography eluting with EtOAc to 7% MeOH in CH₂Cl₂ to provide 5S-[4-(3-chloro-phenyl)-3-hydroxy-butyl]-1-[6-(2H-tetrazol-5-yl)-hexyl]-pyrrolidin-2-one. ¹H NMR (CDCl₃) δ 7.23 (m, 3H), 7.09 (d, 1 H), 3.85(m, 1 H), 3.66 (m, 1 H), 3.53 (m, 1 H), 2.96 (m, 3H), 2.81 (m, 1 H), 2.70 (m, 1 H), 2.44 (m, 21-1), 2.18 (m, 1 H), 1.88-1.27 (m, 14H); MS 420.2 (M+1), 418.3 (M−1).

### EXAMPLE 4

### 5S-[3R-Hydroxy-4-(3-trifluoromethyl-phenyl)-butyl]-1-[6-(2H-tetrazol-5-yl)-hexyl]-pyrrolidin-2-one

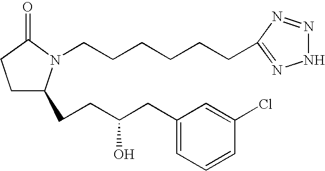

Step A:

7-{2-Oxo-5R-[3-oxo-4-(3-trifluoromethyl-phenyl)-but-1-enyl]-pyrrolidin-1-yl-heptanenitrile

Following the procedure described for Example 1, Step A, the anion 15 derived from [2-oxo-3-(3-trifluoromethyl-phenyl)-propyl]-phosphonic acid dimethyl ester (2.68 g, 8.64 mmol) and NaH (60% in oil, 400 mg, 10 mmol) was reacted with 7-(2R-formyl-5-oxo-pyrrolidin-1-yl)-heptanenitrile (assumed 10 mmol) over 18 h. Purification by medium pressure chromatography (30% EtOAc in hexanes to 80% EtOAc in hexanes) provided 7-{2-oxo-5R-[3-oxo-4-(3-trifluoromethyl-phenyl)-but-1-enyl]-pyrrolidin-1-yl}-heptanenitrile. ¹H NMR (CDCl₃) δ 7.52 (m, 1 H), 7.45 (m, 2H), 7.37 (m, 1 H), 6.67 (dd, 1 H), 6.23 (d, 1 H), 4.18 (m, 1 H), 3.90 (s, 2H), 3.53 (m, 1 H), 2.73 (m, 1 H), 2.45-2.23 (m, 5H), 1.79 (m, 1 H), 1.60 (m, 2H), 1.41 (m, 4H), 1.24 (m, 2H); MS 407.2 (M+1), 405.3 (M−1).

Step B:

7-{2R-[3S-Hydroxy-4-(3-trifluoromethyl-phenyl)-but-1-enyl]-5-oxo-pyrrolidin-1-yl}-heptanenitrile

To a solution of 7-(2-oxo-5R-[3-oxo-4-(3-trifluoromethyl-phenyl) but-1-enyl]-pyrrolidin-1-yl}-heptanenitrile (1.14 g, 2.81 mmol) and (R)-2-methyl CBS-oxazaborolidine (1 M in toluene, 0.42 mL, 0.42 mmol) in CH₂Cl₂ (112 mL) at −45° C. was added catecholborane (1 M in THF, 8.4 mL, 8.4 mmol) dropwise. The reaction mixture was stirred at −45° C. for 18 h and 1 N HCl was added. The reaction mixture was stirred at room temperature for 40 minutes and the layers were separated. The organic solution was washed with cold 1 N NaOH (3 times). The organic solution was washed sequentially with 1 N HCl, water and brine. The organic solution was dried ( MgSO₄), filtered and concentrated. Purification by medium pressure chromatography (1:1 hexanes:EtOAc to 80% EtOAc in hexanes) provided 7-{2R-[3S-hydroxy-4-(3-trifluoromethyl-phenyl)-but-1-enyl]-5-oxo-pyrrolidin-yl}-heptanenitrile as an approximate 2.5:1 ratio of 3S:3R alcohol diastereomers by ¹H NMR. ¹H NMR (CDCl₃) δ 7.51-7.38 (m, 4H), 5.72 (dd, 1 H), 5.49 (dd, 1 H), 4.45 (m, 1 H), 4.02 (m, 1 H), 3.47 (m, 1 H), 2.90 (m, 2H), 2.71 (m, 1 H), 2.34 (m, 4H), 2.18 (m, 1 H), 1.66 (m, 4H), 1.44 (m, 4H), 1.27 (m, 2H); MS 409.2 (M+1).

Step C:

7-{2S-[3R-Hydroxy-4-(3-trifluoromethyl-phenyl)-butyl]-5-oxo-pyrrolidin-1-yl}heptanenitrile

Following the procedure described for Example 1, Step C, 7-{2R[3S-hydroxy-4-(3-trifluoromethyl-phenyl)-but-1-enyl]-5-oxo-pyrrolidin-1-yl}heptanenitrile (810 mg) in MeOH (40 mL) was hydrogenated in the presence of 10% palladium on carbon (100 mg) at 50 psi for 18 h on a Parr shaker. Purification by medium pressure chromatography (1:1 hexanes:EtOAc to EtOAc to 3% MeOH in CH₂Cl₂) provided 7-{2S-[3R-hydroxy-4-(3-trifluoromethyl-phenyl)-butyl]-5-oxo-pyrrolidin-1-yl}-heptanenitrile. ¹H NMR(CDCl₃) δ 7.44(m, 4H), 3.84 (m, 1H), 3.58 (m, 2H), 2.88 (m, 2H), 2.73 (m, 1 H), 2.32 (m, 4H), 2.11 (m, 1 H), 1.78 (m, 1 H), 1.65-1.37 (m, 11 H), 1.30 (m, 2H); MS 411.2 (M+1).

Step D:

5S-[3R-Hydroxy-4-(3-trifluoromethyl-phenyl)-butyl]-1-[6-(2H-tetrazol-5-yl)-Hexyl]-pyrrolidin-2-one

Following the procedure described for Example 3, Step D, 7 2S-(3R-hydroxy-4-(3-trifluoromethyl-phenyl)-butyl]-5-oxo-pyrrolidin-1-yl}heptanenitrile (710 mg, 1.73 mmol) was reacted with azidotrimethylsilane (399 mg, 3.46 mmol) and dibutyltin oxide (43 mg, 1.7 mmol) in toluene (25 mL) heated under reflux for 18 h. The volatiles were removed in vacuo and the residue was diluted with CH₂Cl₂. The organic solution was washed consecutively with 1 N HCl (2 times), water (1 time) and brine (1 time). The organic solution was dried (MgSO₄), filtered, and concentrated. The residue was purified by medium pressure chromatography eluting with EtOAc to 2% MeOH in CH₂Cl₂ to 8% MeOH in CH₂Cl₂ to provide 5S-[3R hydroxy-4-(3-trifluoromethyl-phenyl)-butyl]-1-[6-(2H-tetrazol-5-yl)-hexyl]-pyrrolidin-2-one. ¹H NMR (CDCl₃) δ 7.44 (m, 4H), 3.87 (m, 1 H), 3.65 (m, 1 H), 3.50 (m, 1 H), 3.01-2.73 (m, 5H), 2.42 (m, 2H), 2.16 (m, 1 H), 1.86-1.23 (m, 14H); MS 454.4 (M+1), 452.4 (M−1).

Step E:

Sodium Salt of 5S-[3R-Hydroxy-4-(3-trifluoromethyl-phenyl)-butyl]-1-[6-(2H-tetrazol-5-yl)-hexyl]-pyrrolidin-2-one

Following the procedure described for Example 2, Step E, treatment of 5S-[3R-hydroxy-4-(3-trifluoromethyl-phenyl)-butyl)-1-[6-(2H-tetrazol-5-yl)-hexyl]-pyrrolidin-2-one (428 mg, 0.944 mmol) with NaHCO₃ (79 mg, 0.94 mmol) provided the sodium salt. ¹H NMR (CD₃OD) δ 7.48 (m, 4H), 3.79 (m, 1 H), 3.67 (m, 1 H), 3.51 (m, 1 H), 2.86 (m, 5H), 2.30 (m, 2H), 2.12 (m, 1 H), 1.84-1.27 (m, 14H); MS 454.4 (M-Na+1), 452.4 (M-Na−1).

### EXAMPLE 5

### 5-(4-Biphenyl-3-yl-3-hydroxy-butyl)-1-[6-(2H-tetrazol-5-yl)-hexyl]-pyrrolidin-2-one

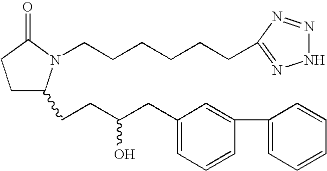

Step A1:

7-{2-[3-(tert-butyl-dimethyl-silanyloxy)-4-(4-fluoro-phenyl)-butyl]-5-oxo-pyrrolidin-1-yl}-heptanenitrile

A solution of 5-[3-(tert-butyl-dimethyl-silanyloxy)-4-(4-fluoro-phenyl)-butyl]-pyrrolidin-2-one (150 mg, 0.41 mmol) in DMF (5 mL) was added to NaH (60% by weight in oil, 16 mg, 0.41 mmol) in DMF (5 mL) was added and the reaction mixture was stirred at 90 C for 2.5 hour. Water (20 mL) was added and the aqueous solution was washed with EtOAc (4×15 mL). The combined organic solutions were washed with water (2×15 mL), dried MgSO₄), filtered, and concentrated. The residue was purified by medium pressure chromatography (1:1 hexanes:EtOAc) provided 7-{2-[3-(tert-butyl-dimethyl-silanyloxy)-4-(4-fluoro-phenyl)-butyl]-5-oxo-pyrrolidin-1-yl}-heptanenitrile. ¹H NMR (CDCl₃) 7.09 (m, 2H), 6.95 (m, 2H), 3.81 (m, 1H), 3.54 (m, 2H), 2.86 (m 1H), 2.68 (m, 2H), 2.29 (m, 4H), 2.06 (m, 1H), 1.74-1.23 (m, 13H), 0.85 (s, 9H), 0.04 (m, 3H); MS 475.1 (M+1).

Step A2:

7-{2-[4-biphenyl-3-yl-3-(tert-butyl-dimethyl-silanyloxy)-butyl]-5-oxo-pyrrolidin-1-yl}-heptanenitrile

Following the procedure described for Step A1 above, the anion derived from 5-[4-biphenyl-3-yl-3-(tert-butyl-dimethyl-silanyloxy)-butyl]-pyrrolidin-2-one (239.1 mg, 0.564 mmol) and NaHMDS (1 M in THF, 0.67 mL, 0.67 mmol) was alkylated with 7-bromoheptanenitrile (118 mg, 0.620 mmol) at 70° C. for 24 h. Purification by medium pressure chromatography (CH₂Cl₂ 1% MeOH in CH₂Cl₂ to 2% MeOH in CH₂Cl₂ to 4% MeOH in CH₂Cl₂) provided 7-{2-[4-biphenyl-3yl-3-(tert-butyl-dimethyl-silanyloxy)-butyl]-5-oxo-pyrrolidin-1-yl}-heptanenitrile. MS 533.3 (M+1).

Step B 1:

7-{2-[4-(4-fluoro-phenyl)-3-hydroxy-butyl]-5-oxo-pyrrolidin-1-yl}-heptanenitrile

To a solution of 7-{2-[3-(tert-butyl-dimethyl-silanyloxy)-4-(4-fluoro-phenyl)-butyl]-5-oxo-pyrrolidin-1-yl}-heptanenitrile (158 mg, 0.333 mmol) in THF (20 mL) at 0 C was added TBAF (1M in THF, 0.50 mL, 0.50 mmol). The reaction mixture was stirred at room temperature for 3 hours and saturated aqueous NaHCO₃ was added. The volatiles were removed in vacuo. The remaining aqueous solution was washed with CHCl₃ (4×5 mL) and the combined organic solutions were dried (MgSO₄), filtered and concentrated. The residue was purified by medium pressure chromatography (1:1 hexanes:EtOAc to EtOAc) provided 7-{2-[4-(4-fluoro-phenyl)-3-hydroxy-butyl]-5-oxo-pyrrolidin-1-yl}heptanenitrile.

Step B2:

7-{2-(4-Biphenyl-3-yl-3-hydroxy-butyl)-5-oxo-pyrrolidin-1-yl]-heptanenitrile

Following the procedure described for Step B 1 above, 7-{2-[4-biphenyl-3-yl-3(tert-butyl-dimethyl-silanyloxy) -butyl]-5-oxo-pyrrolidin-1-yl}-heptanenitrile (187 mg, 0.351 mmol) was deprotected with TBAF (1 M in THF, 0.53 mL, 0.53 mmol). The addition was performed at 0° C. and the reaction mixture was stirred at room temperature for 24 h. Purification by medium pressure chromatography (CH₂Cl₂) to 1% MeOH in CH₂Cl₂ to 2% MeOH in CH₂Cl₂ to 6% MeOH in CH₂Cl₂ to 10% MeOH in CH₂Cl₂) provided 7-[2-(4-biphenyl-3-yl-3-hydroxy-butyl)-5-oxo-pyrrolidin-1-yl]-heptanenitrile. ¹H NMR (CDCl₃) δ 7.58 (m, 1 H), 7.51-7.33 (m, 4H), 7.21-7.12 (m, 4H), 3.85 (m, 1 H), 3.60 (m, 2H), 2.90 (m, 1 H), 2.83-2.60 (m, 2H), 2.45-2.30 (m, 4H), 2.14 (m, 1 H), 1.73-1.25 (m, 14H).

Step C:

5-(4-Biphenyl-3-yl-3-hydroxy-butyl)-1-[6-(2H-tetrazol-5-yl)-hexyl]-pyrrolidin-2-one

Following the procedure described for Example 3, Step D, 7-[2-(4-biphenyl-3-yl-3-hydroxy-butyl)-5-oxo-pyrrolidin-1-yl]-heptanenitrile (109 mg, 0.260 mmol) was reacted with azidotrimethylsilane (0.69 mL, 0.52 mmol) and dibutyltin oxide (11 mg, 0.044 mmol) in toluene (5.3 mL) heated at reflux for 72 h. The reaction mixture was cooled and water was added. The mixture was acidified with 1 N HCl to pH=2 and the aqueous solution was washed with 5% MeOH in CH₂Cl₂ (3 times). The combined organic solutions were dried (MgSO₄), filtered, and concentrated. Purification by medium pressure chromatography (1:1 hexanes:EtOAc to EtOAc to 2% MeOH in CH₂Cl₂ to 4% MeOH in CH₂Cl₂ to 8% MeOH in CH₂Cl₂) provided the title compound of Example 5. ¹H NMR (CDCl₃) δ 7.57 (m, 1 H), 7.51-7.32 (m, 4H), 7.25-7.13 (m, 4H), 3.91 (m, 1 H), 3.74-3.50 (m, 2H), 2.96 (m, 3H), 2.77 (m, 1 H), 2.50 (m, 2H), 2.22 (m, 1 H), 2.07 (m, 1 H), 1.90-1.22 (m, 14H); MS 462.2 (M+1), 460.1 (M−1).

### EXAMPLE 6

### 7-{2R-[3-Hydroxy-4-(3-phenoxy-phenyl)-but-1-enyl]-5-oxo-pyrrolidin-1-yl)-heptanoic Acid

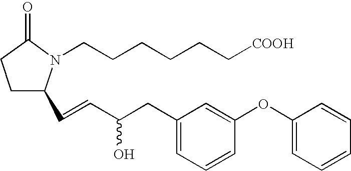

Step A:

7-{2-Oxo-5R-[3-oxo-4-(3-phenoxy-phenyl)-but-1-enyl]-pyrrolidin-1-yl}heptanoic Acid Ethyl Ester

Following the procedure described for Example 1, Step A, the anion derived from [2-oxo-3-(3-phenoxy-phenyl)-propyl]-phosphonic acid dimethyl ester (633 mg, 1.98 mmol) and NaH (60% in oil, 70 mg, 1.74 mmol) was reacted with 7-(2R-formyl-5-oxo-pyrrolidin-1-yl)-heptanoic acid ethyl ester (assumed 1.58 mmol) over 24 h. Medium pressure chromatography (EtOAc) provided 7-(2-oxo-5R-[3-oxo-4-(3-phenoxy-phenyl)-but-1-enyl]-pyrrolidin-1-yl)-heptanoic acid ethyl ester. 1H NMR (CDCl₃) δ 7.28 (m, 3H), 7.08 (m, 1 H), 6.97 (m, 2H), 6.89 (m, 2H), 6.83 (m, 1 H), 6.62 (dd, 1 H), 6.19 (d, 1 H), 4.13 (m, 1 H), 4.08 (q, 2H), 3.79 (s, 2H), 3.51 (m, 1 H), 2.68 (m, 1 H), 2.35 (m, 2H), 2.24 (m, 3H), 2.24 (m, 3H), 1.75 (m, 1 H), 1.54 (m, 2H), 1.43-1.20 (m, 9H).

Step B:

7-{2R-[3-hydroxy-4-(3-phenoxy-phenyl)-but-1-enyl]-5-oxo-pyrrolidin-1-yl}heptanoic Acid Ethyl Ester

Following the procedure described for Example 3, Step C, 7-{2-oxo-5R-[3-oxo-4-(3-phenoxy-phenyl)-but-1-enyl]-pyrrolidin-1-yl)-heptanoic acid ethyl ester (215 mg, 0.451 mmol) was reacted with NaBH₄ (17 mg, 0.45 mmol) in EtOH (3 mL) at 0° C. over 4 h. Purification by medium pressure chromatography (EtOAc) provided 7-{2R-[3-hydroxy-4-(3-phenoxy-phenyl)-but-1-enyl]-5-oxo-pyrrolidin-1-yl)-heptanoic acid ethyl ester. ¹H NMR (CDCl₃) δ 7.33 (m, 2H), 7.25 (m, 1 H), 7.10 (m, 1 H), 6.99 (m, 2H), 6.93 (m, 1 H), 6.86 (m, 2H), 5.72 (m, 1 H), 5.45 (m, 1 H), 4.37 (m, 1 H), 4.10 (q, 2H), 3.47 (m, 1 H), 2.82 (m, 3H), 2.35 (m, 2H), 2.26 (t, 2H), 2.15 (m, 1 H), 1.70-1.21 (m, 13H).

Step C:

7-{2R-[3-Hydroxy-4-(3-phenoxy-phenyl)-but-1-enyl]-5-oxo-pyrrolidin-1-yl)-heptanoic Acid

Following the procedure described for Example 1, Step D, 7-(2R-[3-hydroxy-4-(3-phenoxy-phenyl)-but-1-enyl]-5-oxo-pyrrolidin-1-yl)-heptanoic acid ethyl ester (29 mg, 0.060 mmol) was hydrolyzed with 2M NaOH in EtOH (4.0 mL) at room temperature over 24 h to provide the title compound. ¹H NMR (CDCl₃) δ 7.33-7.21 (m, 3H), 7.08 (m, 1 H), 6.98-6.84 (m, 51-1), 5.70 (m, 1 H), 5.44 (m, 1 H), 4.36 (m, 1 H), 4.00 (m, 1 H), 3.44 (m, 1 H), 2.85-2.51 (m, 3H), 2.32 (m, 41-1), 2.14 (m, 1 H), 1.68-1.18 (m, 10H).

### EXAMPLE 7

### 7-{(2R)-2-[(1E)-3-hydroxy-4-methyl-4-phenylpent-1-enyl]-5-oxopyrrolidin-1-yl}heptanoic Acid

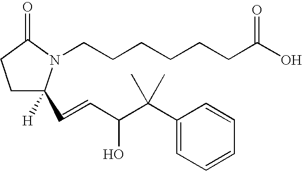

Step A:

Ethyl 7-{(2R)-2-[(1E)-4-methyl-3-oxo-4-phenylpent-1-enyl]-5-oxopyrrolidin-1-yl}heptanoate

To a solution of dimethyl 3-methyl-2-oxo-3-phenylbutylphosphonate (297 mg, 1.1 mmol) in DME (3 ml) at 0° C. was added portionwise NaH 95% (26.4 mg, 1.1 mmol), and the mixture was stirred 20 min at 0° C. Then a solution of 7-(2R-formyl-5-oxo-pyrrolidin-1-yl)-heptanoic acid ethyl ester (269 mg, 1.0 mmol) in DME (2 ml) was added dropwise and the reaction mixture was allowed to reach room temperature, and stirred overnight. A half-saturated solution of NH₄Cl (10 ml) was added and the aqueous phase was extracted with AcOEt (4×60 ml); the organic phases was washed with water (20 ml), brine (20 ml), dried on MgSO₄, filtered and the solvent was removed under reduced pressure. The residual oil was purified by flash column-chromatography on silica gel (eluent AcOEt 1:Hexanes 3 ) to provide ethyl 7-{(2R)-2-[(1E)4-methyl-3-oxo-4-phenylpent-1-enyl]-5-oxopyrrolidin-1-yl}heptanoate as an oil. ¹H NMR (CDCl₃) δ 7.28-7.05 (m, 5H), 6.53 (dd, J=15.5 Hz, 8.0 Hz, 1H), 5.91 (d, J=15.5 Hz, 1H), 4.02 (q, J=7.0 Hz, 2H), 3.90 (m, 1H), 3.25 (m, 1H), 2.41 (m, 1H), 2.30-2.12 (m, 4H), 2.08-1.96 (m, 1H), 1.63-0.95 (m, 18H); MS=414.1 [M+1].

Step B:

Ethyl 7-{(2R)-2-[(1E)-3-hydroxy-4-methyl-4-phenylpent-1-enyl]-5-oxopyrrolidin-1-yl}heptanoate

To a solution of ethyl 7-{(2R)-2-[(1E)-4-methyl-3-oxo-4-phenylpent-1-enyl]-5-oxopyrrolidin-1-yl}heptanoate (240 mg, 0.58 mmol) in (MeOH 5:H₂O 1) (6 ml) at 0° C. was added first CeCl₃.7H₂0 (191.1 mg, 0.777 mmol), and then portionwise NaBH₄ (35.6 mg, 0.93 mmol). The mixture was stirred at 0° C. for 1 h, then addition of 1N HCl (2 ml) and the solvent was removed under reduced pressure. The residue was dissolved in a mixture of water (5 ml) and 1N HCl (1 ml), the aqueous phase was extracted with AcOEt (3×15 ml); the organic phases was washed with water (5 ml), brine (5 ml), dried on MgSO₄, filtered and the solvent was removed under reduced pressure. The residual oil was purified by flash column-chromatography on silica gel (eluent Acetone 3:Toluene 7 ) to provide both diastereoisomers of ethyl 7-{(2R)-2-[(1E)-3-hydroxy-4-methyl-4-phenylpent-1-enyl]-5-oxopyrrolidin-1-yl}heptanoate in a (1:4.5) ratio by NMR as an oil. ¹H NMR (CDCl₃) 7.31-7.13 (m, 5H), 5.47 (m, 1H), 5.34 (m, 1H), 4.14 (m, 1H), 4.03 (q, J=7.1 Hz, 2H), 3.90 (m, 1H), 3.40 (m, 1H), 2.72 and 2.55 (m, 1H), 2.33-2.19 (m, 5H), 2.05 (m, 1H), 1.60-1.15 (m, 18H) ; MS=416.0 [M+1].

Step C:

7-{(2R)-2-[(1E)-3-hydroxy-4-methyl-4-phenylpent-1-enyl]-5-oxopyrrolidin-1-yl}heptanoic Acid

To a solution of ethyl 7-{2R)-2-[(1E)-3-hydroxy-4-methyl-4-phenylpent-1-enyl]-5-oxopyrrolidin-1-yl}heptanoate (87.8 mg, 0.211 mmol) in MeOH/THF (1:3) (4 ml) was added a solution of 1N LiOH (0.422 ml, 0.422 mmol) at 0° C. The reaction mixture is stirred overnight at room temperature, and the solvent was removed under reduced pressure. 0.5N HCl (4 ml) was added, the aqueous phase was extracted with Et₂O (4×10 ml), the organic phases were washed with brine (2 ml), dried on MgSO₄, filtered and the solvent was removed under reduced pressure. The residual oil was purified by flash column-chromatography on silica gel (gradient CH₂Cl₂:MeOH:AcOH (100:0:0) to (95:5:0.5)) to provide both diastereoisomers of 7-{(2R)-2-[(1E)-3-hydroxy-4-methyl-4-phenylpent-1-enyl]-5-oxopyrrolidin-1-yl}heptanoic acid as an oil. ¹H NMR (CDCl₃) δ 7.39-7.16 (m, 5H), 6.6 (sl, 1H), 5.55 (m, 1H), 5.47 (m, 1H), 4.20 (m, 1H), 3.95 (m, 1H), 3.45 (m, 1H), 2.80 and 2.60 (m, 1H), 2.40-2.24 (m, 4H), 2.12 (m, 1H), 1.65-1.13 (m, 16H); MS 386.1 (M−1).

### EXAMPLE 8

### 7-{(2R)-2-[(1E)-3-hydroxy-3-(1-phenylcyclopropyl)prop-1-enyl]-5-oxopyrrolidin-1-yl}heptanoic Acid

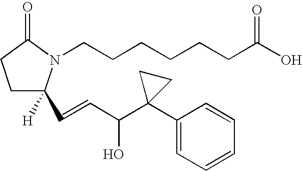

Step A:

Ethyl 7-{(5R)-5-[(1E)-3-oxo-3-(1-phenylcyclopropyl)prop-1-enyl]pyrrolidin-1-yl}heptanoate

To a solution of dimethyl 2-oxo-2-(1-phenylcyclopropyl) ethylphosphonate (270 mg, 1.0 mmol) in DME (3 ml) at 0° C. was added portionwise NaH 95% (24.0 mg, 1.0 mmol), and the mixture was stirred 20 min at 0° C. Then a solution of 7-(2R-formyl-5-oxo-pyrrolidin-1-yl)-heptanoic acid ethyl ester (242.0 mg, 0.90 mmol) in DME (2 ml) was added dropwise and the reaction mixture was allowed to reach room temperature, and stirred overnight. A half-saturated solution of NH₄Cl (10 ml) was added and the aqueous phase was extracted with AcOEt (4×60 ml); the organic phases was washed with water (20 ml), brine (20 ml), dried on MgSO₄, filtered and the solvent was removed under reduced pressure. The residual oil was purified by flash column-chromatography on silica gel (eluent AcOEt 1:Hexanes 3) to provide ethyl 7-{(5R)-5-[(1E)-3-oxo-3-(1-phenylcyclopropyl)prop-1-enyl]pyrrolidin-1-yl}heptanoate as an oil. ¹H NMR (CDCl₃) 7.35-20 (m, 5H), 6.43 (dd, J=15.5 Hz, 8.0 Hz, 1H), 6.05 (d, J=15.5 Hz, 1H), 4.07 (q, J=7.0 Hz, 2H), 3.93 (m, 1H), 3.35 (m, 1H), 2.53 (m, 1H), 2.34-2.16 (m, 4H), 2.12-2.02 (m, 1H), 1.70-1.48 (m, 4H), 1.32-1.10 (m, 12H) ; MS=412.3 [M+1].

Step B:

Ethyl 7-{(2R)-2-[(1E)-3-hydroxy-3-(1-phenylcyclopropyl)prop-1-enyl]-5-oxopyrroidin-1-yl}heptanoate

To a solution of ethyl 7-{(5R)-5-[(1E)-3-oxo-3-(1-phenylcyclopropyl)prop-1-enyl]pyrrolidin-1-yl}heptanoate (295 mg, 0.718 mmol) in (MeOH 5:H₂O) (6 ml) at 0° C. was added first CeCl₃.7H₂O (177.0 mg, 0.718 mmol), and then portionwise NaBH₄ (53.3 mg, 1.436 mmol). The mixture was stirred at 0° C. for 1 h, then the solvent was removed under reduced pressure. The residue was dissolved in a mixture of water (5 ml) and 1N HCl (1 ml), the aqueous phase was extracted with AcOEt (3×15 ml); the organic phases was washed with water (5 ml), brine (5 ml), dried on MgSO₄, filtered and the solvent was removed under reduced pressure. The residual oil was purified by flash column-chromatography on silica gel (eluent AcOEt ) to provide both diastereoisomers of ethyl 7-{(2R)-2-[(1E)-3-hydroxy-3-(1-phenylcyclopropyl)prop-1-enyl]-5-oxopyrrolidin-1-yl}heptanoate in a (1:3.1) ratio by NMR as an oil. ¹H NMR (CDCl₃) 7.29-7.19 (m, 5H), 5.63 (m, 1H), 5.33 (m, 1H), 4.08 (q, J=7.1 Hz, 2H), 3.95 (m, 1H), 3.79 (m, 1H), 3.45 and 3.38 (m, 1H), 2.72 and 2.50 (m, 1H), 2.32-2.23 (m, 4H), 2.12 (m, 1H), 1.98 and 1.90 (m, 1H), 1.60-1.15 (m, 12H), 0.91-0.81 (m, 4H) ; MS=414.4 [M+1].

Step C:

7-{(2R)-2-[(1E)-3-hydroxy-3-(1-phenylcyclopropyl)prop-1-enyl]-5-oxopyrrolidin-1-yl}heptanoic Acid

To a solution of ethyl 7-{(2R)-2-[(1E)-3-hydroxy-3-(1-phenylcyclopropyl)prop-1-enyl]-5-oxopyrrolidin-1-yl}heptanoate (91.4 mg, 0.221 mmol) in MeOH/THF (1:3) (4 ml) was added a solution of 1N LiOH (0.442 ml, 0.442 mmol) at 0° C. The reaction mixture is stirred overnight at room temperature, and the solvent was removed under reduced pressure. 0.5N HCl (4 ml) was added, the aqueous phase was extracted with Et₂O (4×10 ml), the organic phases were washed with brine (2 ml), dried on MgSO₄, filtered and the solvent was removed under reduced pressure to provide both diastereoisomers of 7-{(2R)-2-[(1E)-3-hydroxy-3-(1-phenylcyclopropyl)prop-1-enyl]-5-oxopyrrolidin-1-yl}heptanoic acid as an oil. ¹H NMR (CDCl₃) 7.35-6.95 (m, 6H), 5.62 (m, 1H), 5.30 (m, 1H), 3.95 (m, 1H), 3.80 (m, 1H), 3.45 and 3.85 (m, 1H), 2.72 and 2.60 (m, 1H), 2.40-2.24 (m, 4H), 2.12 (m, 1H), 1.65-1.13 (m, 10H), 0.95-0.75 (m, 4H) ; MS 384.4 (M−1).

### EXAMPLE 9

### 7-{(2R)-2-[(1E)-3-cyclohexyl-3-hydroxyprop-1-enyl]-5-oxopyrrolidin-1-yl}heptanoic Acid

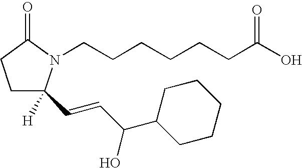

Step A:

Ethyl 7-{(2R)-2-[(1E)-3-cyclohexyl-3-oxoprop-1-enyl]-5-oxopyrrolidin-1-yl}heptanoate

To a solution of dimethyl 3-methyl-2-oxo-3-phenylbutylphosphonate (220 mg, 0.94 mmol) in DME (3 ml) at 0° C. was added portionwise NaH 95% (22.6 mg, 0.94 mmol), and the mixture was stirred 20 min at 0° C. Then a solution of 7-(2R-formyl-5-oxo-pyrrolidin-1-yl)-heptanoic acid ethyl ester (230.0 mg, 0.855 mmol) in DME (2 ml) was added dropwise and the reaction mixture was allowed to reach room temperature, and stirred overnight. A half-saturated solution of NH₄Cl (10 ml) was added and the aqueous phase was extracted with AcOEt (4×60 ml); the organic phases was washed with water (20 ml), brine (20 ml), dried on MgSO₄, filtered and the solvent was removed under reduced pressure. The residual oil was purified by flash column-chromatography on silica gel (eluent AcOEt 1:Hexanes 3 ) to provide ethyl 7-{(2R)-2-[(1E)-3-cyclohexyl-3-oxoprop-1-enyl]-5-oxopyrrolidin-1-yl}heptanoate as an oil. ¹H NMR (CDCl₃) 6.52 (dd, J=15.6 Hz, 7.9 Hz, 1H), 6.14 (d, J=15.6 Hz, 1H), 4.10 (m, 1 H), 3.99 (q, J=7.0 Hz, 2H), 3.47 (m, 11 H), 2.70 (m, 1H), 2.44-2.14 (m, 6H), 1.74-1.67 (m, 4H), 1.59-1.08 (m, 16H); MS=378.6 [M+1].

Step B:

Ethyl 7-{(2R)-2-[(1E)-3-cyclohexyl-3-hydroxyprop-1-enyl]-5-oxopyrrolidin-1-yl}heptanoate

To a solution of ethyl 7-{(2R2-[(1E)-3-cyclohexyl-3-oxoprop-1-enyl]-5-oxopyrrolidin-1-yl}heptanoate (150 mg, 0.398 mmol) in (MeOH 5:H₂O 1) (6 ml) at 0° C. was added first CeCl₃.7H₂O (147.0 mg, 0.597 mmol), and then portionwise NaBH₄ (24.1 mg, 0.637 mmol). The mixture was stirred at 0° C. for 1 h, then the solvent was removed under reduced pressure. The residue was dissolved in a mixture of water (5 ml) and 1N HCl (1 ml), the aqueous phase was extracted with AcOEt (3×15 ml); the organic phases was washed with water (5 ml), brine (5 ml), dried on MgSO₄, filtered and the solvent was removed under reduced pressure. The residual oil was purified by flash column-chromatography on silica gel (eluent AcOEt) to provide both diastereoisomers of ethyl 7-{(2R)-2-[(1E)-3-cyclohexyl-3-hydroxyprop-1-enyl]-5-oxopyrrolidin-1-yl}heptanoate as an oil. ¹H NMR (CDCl₃) 5.64 (m, 1H), 5.42 (m, 1H), 4.08-3.99 (m, 3H), 3.84 (m, 1H), 3.60 (s, 1H), 3.45 (m, 1H), 2.83 (m, 1H), 2.36-2.11 (m, 5H), 1.77-0.88 (m, 23H); MS=380.4 [M+1].

Step C:

7-{(2R)-2-[(1E)-3-cyclohexyl-3-hydroxyprop-1-enyl]-5-oxopyrrolidin-1-yl}heptanoic Acid

To a solution of ethyl 7-{(2R)-2-[(1E)-3-cyclohexyl-3-hydroxyprop-1-enyl]-5-oxopyrrolidin-1-yl}heptanoate (87 mg, 0.229 mmol) in MeOH/THF (1:3) (4 ml) was added a solution of 1N LiOH (0.459 ml, 0.459 mmol) at 0° C. The reaction mixture is stirred overnight at room temperature, and the solvent was removed under reduced pressure. 0.5N HCl (4 ml) was added, the aqueous phase was extracted with Et₂O (4×10 ml), the organic phases were washed with brine (2 ml), dried on MgSO₄, filtered and the solvent was removed under reduced pressure to provide both diastereoisomers of 7-{(2R)-2-[(1E)-3-cyclohexyl-3-hydroxyprop-1-enyl]-5-oxopyrrolidin-1-yl}heptanoic acid as an oil. ¹H NMR (CDCl₃) 6.70 (s1, 1H), 5.68 (m, 1H), 5.45 (m, 1H), 4.05 (m, 1H), 3.88 (m, 1H), 3.48 (m, 1H), 2.88 (m, 1H), 2.45-2.16 (m, 5H), 1.84-0.88 (m, 21H); MS=350.4 (M−1).

### EXAMPLE 10

### 7-{(2R)-2-[(1E)-3-hydroxy-3-methyl-4-phenylbut-1-enyl]-5-oxopyrrolidin-1-yl}heptanoic Acid

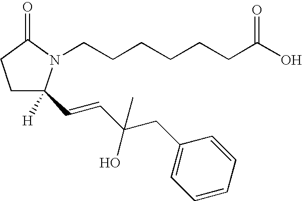

To a solution of anhydrous CeCl₃ (738 mg, 1.87 mmol) and THF (3 ml) was added dropwise MeMgBr (3M in Et2O, 1 ml, 1.0 mmol) at room temperature, and this solution was stirred 12 h. To a solution of ethyl 7-{(2R)-2-[(1E)-3-oxo-4-phenylbut-1-enyl]-5-oxopyrrolidin-1-yl}heptanoate (240 mg, 0.623 mmol) in THF (2 ml) at −78° C. was added the above solution of methylcerium. The mixture was stirred at −78° C. for 4 h. and worked-up with 1N HCl (10 ml). The aqueous phase was extracted with AcOEt (3×30 ml); the organic phases were washed with brine (5 ml), dried on MgSO₄, filtered and the solvent was removed under reduced pressure. The residual oil was partially purified by flash column-chromatography on silica gel (eluent Acetone 3:Toluene 7) to provide both impure diastereoisomers as an oil. To this residue was dissolved in MeOH/THF (3:1) (4 ml) was added a solution of 1N LiOH (0.412 ml, 0.412 mmol) at room temperature. The reaction mixture was stirred overnight at room temperature, and the solvent was removed under reduced pressure. 0.5N HCl (4 ml) was added, the aqueous phase was extracted with Et₂O (4×10 ml), the organic phases were washed with brine (2 ml), dried on MgSO₄, filtered and the solvent was removed under reduced pressure and the residue is purified by flash column-chromatography (gradient (CH₂Cl₂:MeOH:AcOH) from (100:0:0) to (95:5:0.2)) to provide both diastereoisomers of 7-{(2R)-2-[(1E)-3-hydroxy-3-methyl-4-phenylbut-1-enyl]-5-oxopyrrolidin-1-yl}heptanoic acid as an oil. ¹H NMR (CDCl₃) 7.35-7.15 (m, 5H), 5.77 (d, J=14.1 Hz, 1H), 5.34 (m, 1H), 4.03 (m, 1H), 3.45 (m, 1H), 2.85-2.69 (m, 3H), 2.45-2.31 (m, 4H), 2.17 (m, 1H), 1.70-1.21 (m, 14H) ; MS=372.3 (M−1).

### Preparation 1

### 7-(2R-Hydroxymethyl-5-oxo-pyrrolidin-1-yl)-heptanenitrile

Step A:

7-[2R-(tert-Butyl-dimethyl-silanyloxymethyl)-5-oxo-pyrrolidin-1-yl]-Heptanenitrile

To a mixture of NaH (60% in oil, 3.836 g, 0.0959 mmol, washed with 25 mL DMF) in DMF (250 mL) was added a solution of 5R-(tert-butyl-dimethyl silanyloxymethyl)-pyrrolidin-2-one (Tetrahdedron: Asymmetry, 1996, 7, 2113) (20.00 g, 87.19 mmol) in DMF (50 mL). The reaction was stirred at room temperature for 1.5 h and a solution of 7-bromoheptanenitrile (16.574 g, 87.19 mmol) in DMF (50 mL) was added. The reaction was stirred at 90° C. for 3 h. The reaction was cooled to 10 room temperature and water (750 mL) was added. The aqueous solution was washed with EtOAc (4×250 mL). The combined organic solutions were washed with water (2×250 mL), dried (MgSO₄), filtered, and concentrated. Purification by medium pressure chromatography eluting with a solvent gradient (9:1 hexanes:EtOAc to 7:3 hexanes:EtOAc to 1:1 hexanes:EtOAc) provided 7-[2R-(tert-butyl-dimethyl 15 silanyloxymethyl)-5-oxo-pyrrolidin-1-yl)-heptanenitrile. ¹H NMR (CDCl₃) δ 3.69-3.55 (m, 4H), 2.99 (m, 1 H), 2.42 (m, 1 H), 2.34-2.24 (m, 3H), 2.05 (m, 1 H), 1.81 (m, 1 H), 1.67-1.42 (m, 6H), 1.31 (m, 2H), 0.86 (s, 9H), 0.03 (s, 6H); MS 339.3 (M+1).

Step B:

7-(2R-Hydroxymethyl-5-oxo-pyrrolidin-1-yl-heptanenitrile

A solution of tetrabutylammonium fluoride (1M in THF, 100.0 mL, 100.0 mmol) was slowly added to a solution of 7-[2R-(tert-butyl-dimethyl-silanyloxymethyl)-5-oxo-pyrrolidin-1-yl]-heptanenitrile (22.39 g, 66.13 mmol) in THF (400 mL) at 0° C. The reaction was warmed to room temperature and was stirred for 4 h. Saturated aqueous NaHCO₃ (250 mL) was added and the volatiles were removed in vacuo. The remaining aqueous solution was washed with CHCl₃ (4×200 mL). The combined organic solutions were dried (MgSO₄), filtered, and concentrated. Purification by medium pressure chromatography eluting with a solvent gradient (9:1 hexanes:EtOAc to 4:1 hexanes:EtOAc to 7:3 hexanes:EtOAc to 6:4 hexanes:EtOAc to 1:1 hexanes:EtOAc to EtOAc to 9:1 EtOAc:MeOH) provided 7-(2R-hydroxymethyl-5-oxo-pyrrolidin-1-yl)-heptanenitrile. ¹H NMR (CDCl₃) δ 3.78 (dd, 1 H), 3.71-3.58 (m, 3H), 3.00 (m, 1 H), 2.46 (m, 1 H), 2.36-2.27 (m, 3H), 2.08 (m, 1 H), 1.93 (m, 1 H), 1.77 (m, 1 H), 1.68-1.43 (m, 6H), 1.32 (m, 2H); MS 225.1 (M+1).

### Preparation 2

### 7-(2R-Hydroxymethyl-5-oxo-pyrrolidin-1-yl)-heptanoic Acid Ethyl Ester

Step A:

7-[2R-(tert-butyl-dimethyl-silanyloxymethyl)-5-oxo-pyrrolidin-1-yl]-heptanoic Acid Ethyl Ester

Following the procedure described for Preparation 1, Step A, the anion derived from 5R-(tert-butyl-dimethyl-silanyloxymethyl)-pyrrolidin-2-one (30.000 g, 130.8 mmol) and NaH (60% in oil, 5.756 g, 143.9 mmol) in DMF (600 mL) was reacted with ethyl 7-bromoheptanoate (32.559 g, 137.3 mmol) for 3 h at 90° C. Purification by medium pressure chromatography eluting with a solvent gradient (9:1 hexanes:EtOAc to 4:1 hexanes:EtOAc to 7:3 hexanes:EtOAc to 6:4 hexanes:EtOAc) provided 7-[2R-(tert-butyldimethyl-silanyloxymethyl)-5-oxo-pyrrolidin-1-yl]-heptanoic acid ethyl ester. ¹H NMR (CDCl₃) δ 4.10 (q, 2H), 3.62 (m, 4H), 2.95 (m, 1 H), 2.42 (m, 1 H), 2.27 (m, 3H), 2.04 (m, 1 H), 1.81 (m, 1H), 1.65-1.26 (m, 8H), 1.23 (t, 3H), 0.86 (s, 9H), 0.03 (s, 6H); MS 386.2 (M+1).

Step B:

7-(2R-Hydroxymethyl-5-oxo-pyrrolidin-1-yl)-heptanoic Acid Ethyl Ester

Following the procedure described for Preparation 1, Step B, 7-[2R-(tert-butyl-dimethylsilanyloxymethyl)-5-oxo-pyrrolidin-1-yl]-heptanoic acid ethyl ester (39.46 g, 102.3 mmol) was deprotected with TBAF (1 M in THF, 154.0 mL, 154.0 mmol) with a reaction time of 2.5 h. Purification by medium pressure chromatography eluting with a solvent gradient (9:1 hexanes:EtOAc to 6:4 hexanes:EtOAc to 1:1 hexanes:EtOAc to EtOAc to 19:1 20 EtOAc:MeOH) provided 7-(2R-hydroxymethyl-5-oxo-pyrrolidin-1-yl)-heptanoic acid ethyl ester. ¹ H NMR (CDCl₃) δ 4.10 (q, 2H), 3.77 (dd, 1 H), 3.64 (m, 3H), 2.96 (m, 1 H), 2.46 (m, 1 H), 2.35-2.25 (m, 3H), 2.08 (m, 1 H), 1.93 (m, 1 H), 1.71 (m, 1 H), 1.63-1.27 (m, 8H), 1.23 (t, 3H); MS 272.2 (M+1).

### Preparation 3

### [2-Oxo-3-(3-trifluoromethyl-phenyl)-propyl]-phosphonic Acid Dimethyl Ester

Step A:

N-Methoxy-N-methyl-2-(3-trifluoromethyl-phenyl)-acetamide.

To a solution of N,O-dimethylhydroxylamine hydrochloride (1.577 g, 16.2 mmol) in DMF (25 mL) and CH₂Cl₂ (25 mL) at 0° C. was added triethylamine (2.25 mL). After stirring for 5 minutes, 3-trifluoromethylphenyl acetic acid (3.0 g, 14.7 mmol), HOBT (3.177 g, 23.5 mmol), and DEC (2-diethylaminoethyl chloride hydrochloride, 3.10 g, 16.2 mmol) were added. The reaction mixture was stirred at room temperature for 18 h and was concentrated in vacuo. The residue was diluted with EtOAc and the organic solution was washed consecutively with 1 N NaOH (2 times), water, and brine. The organic solution was dried (MgSO₄), filtered and concentrated in vacuo. Medium pressure chromatography (20% EtOAc in hexanes to 50% EtOAc in hexanes) provided N-methoxy-N-methyl-2-(3-trifluoromethyl-phenyl)-acetamide.

Step B:

[2-Oxo-3-(3-trifluoromethyl-phenyl)-propyl)phosphonic Acid Dimethyl Ester

To a solution of dimethyl methylphosphonate (9.4 g, 75.8 mmol) in toluene (80 mL) at −78° C. was slowly added n-BuLi (2.5M in hexanes, 28 mL, 70 mmol). The reaction mixture was stirred for 1 h and a solution of N-methoxy-N-methyl-2-(3-trifluoromethyl-phenyl)-acetamide (14.39 g) in toluene (50 mL) was slowly added. The reaction mixture was stirred for 2.5 h and AcOH (40 mL) was added. The reaction mixture was warmed to room temperature and water was added. The organic layer was washed with water followed by brine. The organic solution was dried (MgSO₄), filtered and concentrated in vacuo. Medium pressure chromatography (CH₂Cl₂ to 2% MeOH in CH₂Cl₂ ) provided the title compound of Preparation 3. ¹H NMR (CDCl₃) δ 7.52 (m, 1 H), 7.44 (m, 2H), 7.37 (m, 1 H), 3.96 (s, 2H), 3.87 (s, 3H), 3.76 (s, 15 3H), 3.12 (d, 2H).

### Preparation 4

### [3-(3-Chloro-phenyl)-2-oxo-propyl)-phosphonic Acid Dimethyl Ester

Substituting the appropriate starting materials, the title compound of Preparation 4 was prepared following an analogous procedure to that described for Preparation 3.

### Preparation 5

### [3-(3-Chloro-phenyl)-2-oxo-propyl]-phosphonic Acid Dimethyl Ester

To a solution of dimethyl methylphosphonate (17.93 g, 144 mmol) in THF (270 mL) at −78° C. was slowly added n-BuLi (2.5M, 64.2 mL, 160.6 mmol). The reaction mixture was stirred for 1 h and (3-chloro-phenyl)-acetic acid methyl ester (26.93 g, 146 mmol) was slowly added. The reaction mixture was allowed to warm to room temperature and was stirred for 24 h. AcOH (15 mL) was added and the volatiles were removed in vacuo. The residue was diluted with CH₂Cl₂ and the organic solution was washed carefully with saturated aqueous NaHCO₃ (3 times). The organic layer was dried (MgSO₄), filtered and concentrated in vacuo. Purification by medium pressure chromatography (20% EtOAc in hexanes to EtOAc) provided the title compound.

### Preparation 6

### Tetrahydro-pyrolizine-3,5-dione

The title compound of Preparation 6 was prepared following the procedure described in U.S. Pat. No. 4,663,464.

### Preparation 7

### 7-(2R-Formyl-5-oxo-pyrrolidin-1-yl)-heptanoic Acid Ethyl Ester

To a solution of 7-(2R-hydroxymethyl-5-oxo-pyrrolidin-1-yl)-heptanoic acid ethyl ester (1.63 g, 6.01 mmol) in benzene (50 mL) was added -(3-dimethylaminopropyl)-3 ethylcarbodiimide hydrochloride (3.46 g, 18.03 mmol) and DMSO (1.5 mL, 24.04 10 mmol). The solution was cooled to O° C. and pyridinium trifluoroacetate (1.28 g, 6.61 mmol) was added. The reaction mixture was stirred at 0° C. for 15 minutes and then at room temperature for 2 h. The solution was decanted from the oily residue. The residue was washed with benzene (3×) and the combined benzene washes were concentrated in vacuo to provide 7-(2R-formyl-5-oxo-pyrrolidin-1-yl)-heptanoic acid ethyl ester, which was used without further purification.

### Preparation 8

### 4-(3-{2-[4-Biphenyl-3-yl-3-(tert-butyl-dimethyl-silanyloxy)-butyl]-5-oxo-pyrrolidin-1-yl}propyl)-benzoic Acid Methyl Ester

Step A:

5-(3-Bromo-3-oxo-butyl)-pyrrolidin-2-one

To a solution of tetrahydropyrrolizine-3,5-dione (5 g, 36 mmol) in CH₂Cl₂ (320 mL) at O° C. was added 3-bromobenzyl magnesium bromide (0.25M in Et₂O, 155 mL, 38.8 mmol) dropwise. The solution was stirred at O° C. for 2 h and was quenched with saturated aqueous ammonium chloride. Aqueous 1 N HCl was added to achieve a pH=3. After warming to room temperature, the aqueous solution was extracted with CH₂Cl₂ (3×). The combined organic extracts were dried (MgSO₄), filtered, and concentrated. Purification by medium pressure chromatography using a solvent gradient (1:1 hexanes:EtOAc to EtOAc to 5% MeOH in CH₂Cl₂ t) provided 5-(3-bromo-3-oxo-butyl)pyrrolidin-2-one. ¹ H NMR (CDCl₃) δ 7.41-7.11 (m, 4H), 6.24 (bs, 1 H), 3.67 (s, 2H), 3.60 (m, 1 H), 2.52 (t, 2H), 2.32 (m, 2H), 2.20 (m, 1 H), 1.88-1.60 (m, 3H).

Step B:

5-(3-Bromo-3-hydroxy-butyl)-pyrrolidin-2-one

To a solution of 5-(3-bromo-3oxo-butyl)-pyrrolidin-2-one (7.84 g, 25.3 mmol) in EtOH (130 ml-) at O° C. was added NaBH₄ (480 mg, 12.6 mmol) and the reaction was stirred at O° C. for 2.5 h. The reaction was quenched with saturated aqueous ammonium chloride. Water and CH₂Cl₂ were added. The aqueous layer was washed with CH₂Cl₂ (3×) and the combined organic extracts were dried (MgSO₄), filtered, and concentrated. Purification by medium pressure chromatography using a solvent gradient (1:1 hexanes:EtOAc to EtOAc to 1% MeOH in CH₂Cl₂ to 3% MeOH in CH₂Cl₂ to 5% 5 MeOH in CH₂Cl₂ to 8% MeOH in CH₂Cl₂) provided 5-(3-bromo-3-hydroxy-butyl)pyrrolidin-2-one. ¹H NMR (CDCl₃) δ 7.36-7.09 (m, 4H), 6.27 (d, 1 H), 3.78 (m, 1 H), 3.63 (m, 1 H), 2.75 (m, 1 H), 2.62 (m, 1 H), 2.32-2.18 (m, 3H), 1.88 (m, 1 H), 1.731.42 (m, 5H); MS 312.2, 314.1 (M+).

Step C:

5-[3-Bromo-3-(tert-butyl-dimethyl-silanyloxy)-butyl]-pyrrolidin-2-one

To a solution of 5-(3-bromo-3-hydroxy-butyl)-pyrrolidin-2-one (6.76 g, 21.6 mmol) in DMF (86 mL) was added tert-butyldimethylsilyl chloride (3.59 g, 23.8 mmol) followed by imidazole (2.95 g, 43.3 mmol) and DMAP (264 mg, 2.16 mmol). The reaction was stirred for 24 h and was quenched with saturated aqueous ammonium chloride. The aqueous solution was washed with EtOAc (3×) and the combined organic extracts were dried (MgSO₄), filtered, and concentrated. Purification by medium pressure chromatography using a solvent gradient (CH₂Cl₂ to 1% MeOH in CH₂Cl₂ to 3% MeOH in CH₂Cl₂ to 5% MeOH in CH₂Cl₂ to 8% MeOH in CH₂Cl₂) provided 5-[3 bromo-3-(tert-butyl-dimethyl-silanyloxy)-butyl]-pyrrolidin-2-one. 1 H NMR (CDCl₃) δ 7.30 (m, 2H), 7.12 (m, 1 H), 7.04 (m, 1 H), 5.71 (m, 1 H), 3.81 (m, 1 H), 3.56 20 (m, 1 H), 2.66 (m, 2H), 2.32-2.17 (m, 3H), 1.70-1.35 (m, 5H), 0.82 (s, 9H), −0.06 (d, 3H), −0.24 (d, 3H); MS 426.2,428.2 (M+).

Step D:

5-[4-Biphenyl-3-yl-3-(tert-butyl-dimethyl-silanyloxy)-butyl]-pyrrolidin-2-one

To a solution of 5-[3-bromo-3-(tert-butyl-dimethyl-silanyloxy)-butyl]-pyrrolidin-2-one (750 mg, 1.76 mmol) in DME (15 mL) was added phenylboronic acid (236 mg, 1.93 25 mmol). Palladium acetate (26.8 mg, 0.088 mmol) and tri-o-tolylphosphine (39.5 mg, 0.176 mmol) were added followed by a solution of Na₂CO₃ (37.3 mg, 3.52 mmol) in water (1.8 mL). The reaction was heated at reflux for 24 h. The reaction was cooled and the volatiles were removed in vacuo. The residue was diluted with brine and EtOAc. The aqueous solution was washed with EtOAc (3×) and the combined organic extracts were dried (MgSO₄), filtered, and concentrated. Purification by medium pressure chromatography eluting with a solvent gradient (1:1 hexanes:EtOAo to EtOAc to 1% MeOH in CH₂Cl₂ to 3% MeOH in CH₂Cl₂ to 5% MeOH in CH₂Cl₂) provided 5-[4-biphenyl-3-yl-3-(tert-butyl-dimethyl-silanyloxy)-butyl]-pyrrolidin-2-one. ¹H NMR (CDCl₃) δ 7.57 (m, 2H), 7.43 (m, 21-1), 7.33 (m, 3H), 7.11 (m, 2H), 5.78 (m, 1 H), 3.91 (m, 1 H), 3.59 (m, 1 H), 2.76 (m, 2H), 2.27 (m, 3H), 1.73-1.38 (m, 5H), 0.83 (s, 9H), −0.03 (d, 5H), −0. 16 (d, 3H); MS 424.3 (M+1).

### Preparation 9

### 5-[3-(tert-Butyl-dimethyl-silanyloxy)-4-(3-fluoro-phenyl)-butyl]-pyrrolidin-2-one

Step A:

5-[4-(3-Fluoro-phenyl)-3-oxo-butyl]-pyrrolidin-2-one

Following the procedure described for Preparation 8, Step A, tetrahydro-pyrrolizine-3,5-dione (2 g, 14 mmol) was reacted with 3-fluorobenzyl magnesium chloride (0.25M in Et₂O, 62 mL, 15.5 mmol) over 2.5 h. Purification by medium pressure chromatography using a solvent gradient (1:1 hexanes:EtOAc to 2:1 EtOAc:hexanes to EtOAc to 2% MeOH in CH₂Cl₂ to 10% MeOH in CH₂Cl₂) provided 5-[4-(3-fluoro-phenyl)-3-oxo-butyl]-pyrrolidin-2-one. ¹H NMR (CDCl₃) δ 7.32-7.27 (m, 1 H), 7.00-6.90 (m, 3H), 6.12 (bs, 1 H), 3.69 (s, 2H), 3.59 (m, 1 H), 2.52 (t, 2H), 2.30 (m, 2H), 2.19 (m, 1 H), 1.75 (m, 2H), 1.65 (m, 1 H).

Step B:

5-[4-(3-Fluoro-phenyl)-3-hydroxy-butyl]-pyrrolidin-2-one

Following the procedure described for Preparation 8, Step B, 5-[4-(3-fluoro-phenyl)-3-oxo-butyl]-pyrrolidin-2-one (2.17 g, 8.71 mmol) was reduced with NaBH₄ (165 mg, 4.35 mmol). Purification by medium pressure chromatography using a solvent gradient (1:1 hexanes:EtOAc to EtOAc to 1% MeOH in CH₂Cl₂ to 3% MeOH in CH₂Cl₂ to 6% MeOH in CH₂Cl₂) provided 5-[4-(3-fluoro-phenyl)-3-hydroxy-butyl]-pyrrolidin-2-one. ¹H NMR (CDCl₃) δ 7.27 (m, 1 H), 6.94 (m, 3H), 6.38 (m, 1 H), 3.82 (m, 1 H), 3.66 (m, 1 H), 2.79 (m, 1 H), 2.67 (m, 1 H), 2.33-2.21 (m, 3H), 1.92 (d, J=4.15 Hz, 1H), 1.75-1.40 (m, 5H); MS 252.2 (M+1).

Step C:

5-[3-(tert-Butyl-dimethyl-silanyloxy)-4-(3-fluoro-phenyl)-butyl]-pyrrolidin-2-one

Following the procedure described for Preparation 8, Step C, 5-[4-(3-fluoro-phenyl)-3-hydroxy-butyl]-pyrrolidin-2-one (2.23 g, 8.87 mmol) was reacted with tertbutyldimethylsilyl chloride (1.47 g, 9.76 mmol). Purification by medium pressure chromatography using a solvent gradient (1:1 hexanes:EtOAc to EtOAc to 1% MeOH in CH₂Cl₂ to 2% MeOH in CH₂Cl₂ to 4% MeOH in CH₂Cl₂) provided 5-[3-(tert-butyldimethyl-silanyloxy)-4-(3-fluoro-phenyl)-butyl]-pyrrolidin-2-one. ¹H NMR (CDCl₃) δ 7.23 (m, 1 H), 6.88 (m, 3H), 5.75 (m, 1 H), 3.85 (m, 1 H), 3.57 (m, 1 H), 2.71 (m, 2H), 2.30 (m, 2H), 2.25 (m, 1 H), 1.70-1.38 (m, 5H), 0.84 (s, 9H), 0 (s, 3H), −0.2 (s, 3H).

### Preparation 10

### 5-[3-(tert-Butyl-dimethyl-silanyloxy)-4-(4-fluoro-phenyl)-butyl]-pyrrolidin-2-one

Step A:

5-[4-(4-Fluoro-phenyl)-3-oxo-butyl]-pyrrolidin-2-one

Following the procedure described for Preparation 8, Step A, tetrahydro-pyrrolizine-3,5-dione (1.41 g, 10.15 mmol) was reacted with 4-fluorobenzyl magnesium chloride (0.25M in Et₂O, 50 mL, 12.5 mmol) over 5 h. Purification by medium pressure chromatography (2% MeOH in CH₂Cl₂) provided 5-[4-(4-fluoro-phenyl)-3-oxo-butyl]-pyrrolidin-2-one. ¹H NMR (CDCl₃) δ 7.18 (m, 2H), 7.03 (m, 2H), 6.34 (m, 1 H), 3.70 (s, 2H), 3.62 (m, 1 H), 2.54(t, 2H), 2.34-2.15 (m, 3H), 1.82-1.61 (m, 3H).

Step B:

5-[4-(4-Fluoro-phenyl)-3-hydroxy-butyl]-pyrrolidin-2-one

Following the procedure described for Preparation 8, Step B, 5-[4-(4-fluoro-phenyl)-3-oxo-butyl]-pyrrolidin-2-one (2.64 g, mmol) was reduced with NaBH₄ (400 mg, mmol) at room temperature for 1 h. Additional NaBH₄ (150 mg) was added and the reaction was stirred for 20 h. Purification by medium pressure chromatography using a solvent gradient (CH₂Cl₂to 2% MeOH in CH₂Cl₂ to 4% MgOH in CH₂Cl₂) provided 5-[4-(4-fluoro-phenyl)-3-hydroxy-butyl]-pyrrolidin-2-one. ¹H NMR (CDCl₃) δ 7.14 (m, 2H), 6.98 (m, 2H), 6.78 (m, 1 H), 3.76 (m, 1 H), 3.65 (m, 1 H), 2.76 (m, 1 H), 2.64 (m, 1H), 2.32-2.18 (m, 4H), 1.72-1.47 (m, 5H).

Step C:

5-[3-(tert-Butyl-dimethyl-silanyloxy)-4-(4-fluoro-phenyl)-butyl]-pyrrolidin-2-one

Following the procedure described for Preparation 8, Step C, 5-[4-(4-fluoro-phenyl)-3-hydroxy-butyl]-pyrrolidin-2-one (1.95 g, 7.79 mmol) was reacted with tertbutyldimethylsilyl chloride (1.47 g, 9.76 mmol). Purification by medium pressure chromatography (1% MeOH in CH₂Cl₂) provided 5-[3-(tert-butyl-dimethyl-silanyloxy)-4-(4-fluoro-phenyl)-butyl]-pyrrolidin-2-one. ¹H NMR (CDCl₃) δ 7.12 (m, 2H), 6.97 (m, 2H), 5.75 (m, 1 H), 3.83 (m, 1 H), 3.60 (m, 1 H), 2.71 (m, 2H), 2.36-2.24 (m, 3H), 1.70-1.38 (m, 5H), 6.84 (s, 9H), −0.05 (d, 3H), −0.2 (d, 3H).

### Preparation 11

### [2-Oxo-3-(3-phenoxy-phenyl)-propyl]-phosphonic Acid Dimethyl Ester

Substituting the appropriate starting materials, the title compound of Preparation 11 was made in an analogous manner to that described for the compound of Preparation 5.

I. Effects of an EP4 Agonist on Intraocular Pressure (IOP) in Rabbits and Monkeys.

Animals

Drug-naïve, male Dutch Belted rabbits and female cynomolgus monkeys are used in this study. Animal care and treatment in this investigation are in compliance with guidelines by the National Institute of Health (NIH) and the Association for Research in Vision and Ophthalmology (ARVO) resolution in the use of animals for research. All experimental procedures are approved by the Institutional Animal Care and Use Committee of Merck and Company.

Drug Preparation and Administration

Drug concentrations are expressed in terms of the active ingredient (base). The compounds of this invention are dissolved in physiological saline at 0.01, 0.001, 0.0001% for rabbit study and 0.05, 0.005% for monkey studies. Drug or vehicle aliquots (25 ul) are administered topically unilaterally or bilaterally. In unilateral applications, the contralateral eyes receive an equal volume of saline. Proparacaine (0.5%) is applied to the cornea prior to tonometry to minimize discomfort. Intraocular pressure (IOP) is recorded using a pneumatic tonometer (Alcon Applanation Pneumatonograph) or equivalent.

Statistical Analysis

The results are expressed as the changes in IOP from the basal level measured just prior to administration of drug or vehicle and represent the mean, plus or minus standard deviation. Statistical comparisons are made using the Student's t-test for non-paired data between responses of drug-treated and vehicle-treated animals and for paired data between ipsilateral and contralateral eyes at comparable time intervals. The significance of the date is also determined as the difference from the “t-0” value using Dunnett's “t” test. Asterisks represent a significance level of p<0.05.

A. Intraocular Pressure Measurement in Rabbits

Male Dutch Belted rabbits weighing 2.5-4.0 kg were maintained on a 12-hour light/dark cycle and rabbit chow. All experiments were performed at the same time of day to minimize variability related to diurnal rhythm. IOP was measured before treatment then the compound of Example 4 or vehicle were instilled (one drop of 25 ul) into one or both eyes and IOP was measured at 30, 60, 120, 180, 240, 300, and 360 minutes after instillation. In some cases, equal number of animals treated bilaterally with vehicle only were evaluated and compared to drug treated animals as parallel controls.

B. Intraocular Pressure Measurements in Monkeys.

Unilateral ocular hypertension of the right eye was induced in female cynomolgus monkeys weighing between 2 and 3 kg by photocoagulation of the trabecular meshwork with an argon laser system (Coherent NOVUS 2000, Palo Alto, USA) using the method of Lee at al. (1985). The prolonged increase in intraocular pressure (IOP) resulted in changes to the optic nerve head that were similar to those found in glaucoma patients.

For IOP measurements, the monkeys wee kept in a sitting position in restraint chairs for the duration of the experiment. Animals were lightly anesthetized by the intramuscular injection of ketamine hydrochloride (3-5 mg/kg) approximately five minutes before each IOP measurement and one drop of 0.5% proparacaine was instilled prior to recording IOP. IOP was measured using a pneumatic tonometer (Alcon Applanation Tonometer) or a Digilab pneumatonometer (Bio-Rad Ophthalmic Division, Cambridge, Mass., USA).

IOP was measured before treatment and generally at 30, 60, 124, 180, 300, and 360 minutes after treatment. Baseline values were also obtained at these time points generally two or three days prior to treatment. Treatment consisted of instilling one drop of 25 ul of the compound of example 4 (0.05 and 0.005%) or vehicle (saline). At least one-week washout period was employed before testing on the same animal. The normotensive (contralateral to the hypertensive) eye was treated in an exactly similar manner to the hypertensive eye. IOP measurements for both eyes were compared to the corresponding baseline values at the same time point. Results were expressed as mean plus-or-minus standard deviation in mm Hg. The activity range of the compounds of this invention for ocular use is between 0.01 and 100,000 nM

II. Radioligand Binding Assays:

The assays used to test these compounds were performed essentially as described in: Abramovitz M, Adam M, Boie Y, Carriere M, Denis D, Godbout C, Lamontagne S, Rochette C, Sawyer N, Tremblay N M, Belley M, Gallant M, Dufresne C, Gareau Y, Ruel R, Juteau H, Labelle M, Ouimet N, Metters K M. The utilization of recombinant prostanoid receptors to determine the affinities and selectivities of prostaglandins and related analogs. Biochim Biophys Acta 2000 Jan. 17;1483(2):285-293 and discussed below:

Stable Expression of Prostanoid Receptors in the Human Embryonic Kidney (HEK) 293(EBNA) Cell Line

Prostanoid receptor (PG) cDNAs corresponding to full length coding sequences were subcloned into the appropriate sites of the mammalian expression vector pCEP4 (Invitrogen). pCEP4PG plasmid DNA was prepared using the Qiagen plasmid preparation kit (QIAGEN) and transfected into HEK 293(EBNA) cells using LipofectAMCaE@ (GIBCO-BRL) according to the manufacturers' instructions. HEK 293(EBNA) cells expressing the cDNA together with the hygromycin resistance gene were selected in Dulbecco's Modified Eagle Medium (DMEM) supplemented with 10% heat inactivated fetal bovine serum, 1 mM sodium pyruvate, 100 U/ml Penicillin-G, 100 μg/ml Streptomycin sulphate, 250 μg/ml active GENETICIN™ (G418) (all from Life Technologies, Inc./BRL) and 200 μg/ml hygromycin (Calbiochem). Individual colonies were isolated after 2-3 weeks of growth under selection using the cloning ring method and subsequently expanded into clonal cell lines. Expression of the receptor cDNA was assessed by receptor binding assays.

HEK 293(EBNA) cells were grown in supplemented DMEM complete medium at 37° C. in a humidified atmosphere of 6% CO₂ in air, then harvested and membranes prepared by differential centrifugation (1000×g for 10 min, then 160,000×g for 30 min, all at 4° C.) following lysis of the cells by nitrogen cavitation at 800 psi for 30 min on ice in the presence of protease inhibitors (2 mM phenylmethylsulfonylfluoride, 10 μM E-64, 100 μM leupeptin and 0.05 mg/ml pepstatin). The 160,000×g pellets were resuspended in 10 mM HEPES/KOH (pH 7.4) containing 1 mM EDTA at approximately 5-10 mg/ml protein by Dounce homogenisation (Dounce A; 10 strokes), frozen in liquid nitrogen and stored at −80° C.

Prostanoid Receptor Binding Assays

Prostanoid receptor binding assays were performed in a final incubation volume of 0.2 ml in 10 mM MES/KOH (pH 6.0) (EP subtypes, FP and TP) or 10 mM HEPES/KOH (pH 7.4) (DP and IP), containing 1 mM EDTA, 10 mM MgCl₂ (EP subtypes) or 10 mM MnCl₂ (DP, FP, IP and TP) and radioligand [0.5-1.0 nM [³H]PGE₂ (181 Ci/mmol) for EP subtypes, 0.7 nM [³ HPGD₂ (115 Ci/mmol) for DP, 0.95 nM [³H]PGF_{2α} (170 Ci/mmol) for FP, 5 nM [³H]iloprost (16 Ci/mmol) for IP and 1.8 nM [³H]SQ 29548 (46 Ci/mmol) for TP]. EP₃ assays also contained 100 μM GTPγS. The reaction was initiated by addition of membrane protein (approximately 30 μg for EP₁, 20 μg for EP₂, 2 μg for EP₃, 10 μg for EP₄, 60 μg for FP, 30 μg for DP, 10 μg for IP and 10 μg for TP) from the 160,000×g fraction. Ligands were added in dimethylsulfoxide (Me₂SO) which was kept constant at 1% (v/v) in all incubations. Non-specific binding was determined in the presence of 1 μM of the corresponding non-radioactive prostanoid. Incubations were conducted for 60 min (EP subtypes, FP and IP) or 30 min (DP and TP) at 30° C. (EP subtypes, DP, FP and TP) or room temperature (IP) and terminated by rapid filtration through a 96-well Unifilter GF/C (Canberra Packard) prewetted in assay incubation buffer without EDTA (at 4° C.) and using a Tomtec Mach III 96-well semi-automated cell harvester. The filters were washed with 3-4 ml of the same buffer, dried for 90 min at 55° C. and the residual radioactivity bound to the individual filters determined by scintillation counting with addition of 50 μl of Ultima Gold F (Canberra Packard) using a 1450 MicroBeta (Wallac). Specific binding was calculated by subtracting non-specific binding from total binding. Specific binding represented 90-95% of the total binding and was linear with respect to the concentrations of radioligand and protein used. Total binding represented 5-10% of the radioligand added to the incubation media.

The activity range of the compounds of this is between 0.01 and 100,000 nM.

What is claimed is:

1. A method for treating disorders related to elevated intraocular pressure by: treating ocular hypertension, treating glaucoma, treating macular edema, treating macular degeneration, increasing retinal and optic nerve head blood velocity, increasing retinal and optic nerve tension, providing a neuroprotective effect or treating dry eyes, comprising administration to a patient in need of such treatment a therapeutically effective amount of a compound of structural formula Ia, Ib, Ic, Id, Ie or If: 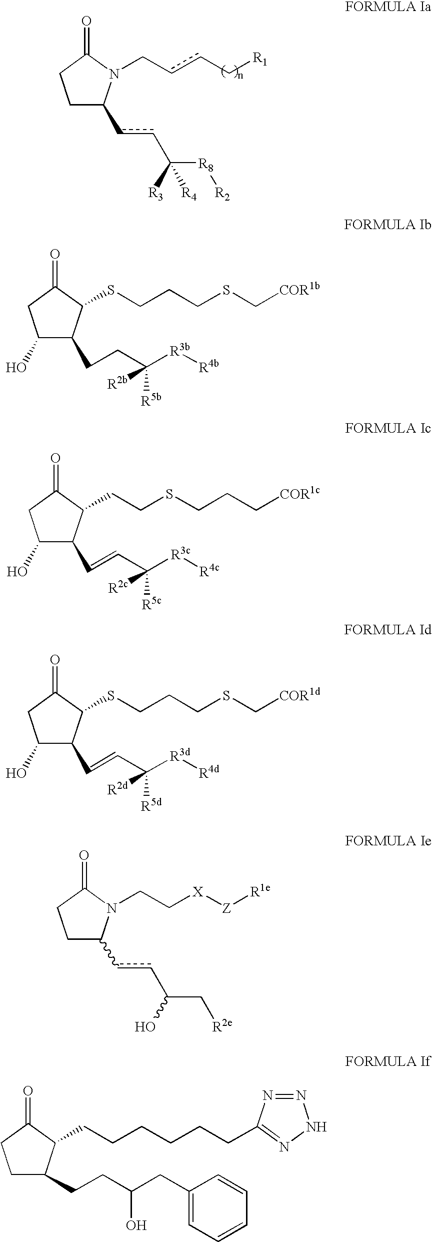
or a pharmaceutically acceptable salt, cyclodextrin clathrate, enantiomer, diastereomer or mixture thereof:
wherein,
R₁ represents COOR₅, CONHR₆ or tetrazol-5-yl;
R₃ and R₄ are each independently hydrogen, hydroxy or C₁₋₃ alkyl;
R₂ represents hydrogen, α-thienyl, phenyl or phenoxy, wherein said phenyl and phenoxy are optionally substituted with 1-3 substituents selected from chloro, fluoro, phenyl, methoxy, trifluoromethyl or C₁₋₃ alkyl; [symbol]
represents a single or double bond;
n is 0to 3;
R₅ represents hydrogen, C₁₋₅ alkyl, phenyl or p-biphenyl;
R₆ represents COR₇ or SO₂R₇;
R₇ represents phenyl or C₁₋₅ alkyl;
R₈ represents C₃₋₆ cycloalkyl or C₁₋₆ alkyl, wherein said cycloalkyl and alkyl groups are optionally substituted with one or two C₁₋₆ alkyl groups;
R^{1b} represents hydroxy, C₁₋₆ alkyloxy or NR^{6b}R^{7b}, wherin R^{6b} and R^{7b} are each independently hydrogen or C₁₋₆ alkyl;
R^{2b} represents hydrogen or hydroxy;
R^{3b} represents a single bond or C₁₋₆ alkylene;
R^{4b} represents
(i) C₁₋₈ alkyl, C₂₋₈ alkenyl, C₂₋₈ alkynyl, phenyl or cycloalkyl, wherein said alkyl, alkenyl, alkynyl are optionally substituted with phenyl, cycloalkyl, OH, C₁₋₆ alkyloxy or halogen;
(ii) Phenyloxy or C₃₋₇ cycloalkyloxy;
(iii) Furyl, furyloxy, thienyl, thienyloxy, naphthyl, naphthyloxy, phthalanyl or phthalanyloxy;
(iv) Phenyl, phenyloxy, C₃₋₇ cycloalkyl or C₃₋₇ cycloalkyloxy, wherein said phenyl, phenyloxy, cycloalkyl and cycloalkyloxy groups are optionally substituted with 1-3 substituents selected from C₁₋₆ alkyl, C₂₋₆ alkenyl, C₂₋₆ alkynyl, C₁₋₆ alkyloxy, C₁₋₆ alkyloxy-C₁₋₆ alkyl, C₁₋₆ alkyloxy-C₁₋₆ alkyloxy, C₁₋₆ alkenyloxy-C₁₋₆ alkyl, C₁₋₆ alkyl substituted by 1-3 hydroxy or halogen groups, C₁₋₆ alkylthio, C₁₋₆ alkylthio-C₁₋₆ alkyl, C₁₋₆ alkylthio-C₁₋₆ alkyloxy, C₁₋₆ alkenylthio-C₁₋₆ alkyl, C₁₋₆ alkylsulfonyl, halogen, trihalomethyl, cyano, nitro, amino, OH, C₃₋₇ cycloalkyl, C₃₋₇ cycloalkyloxy, C₃₋₇ cycloalkyl-C₁₋₆ alkyl, C₃₋₇ cycloalkyloxy-C₁₋₆ alkyl, phenyl, phenyloxy, phenyl-C₁₋₆ alkyl, phenyl-C₂₋₆ alkenyl, phenyl-C₂₋₆ alkynyl, phenyloxy-C₁₋₆ alkyl, phenyloxy-C₂₋₆ alkenyl, phenyloxy-C₂₋₆ alkynyl, furyl, furyloxy, furyl-C₁₋₆ alkyl, furyloxy-C₁₋₆ alkyl, thienyl, thienyloxy, thienyl-C₁₋₆ alkyl or thienyloxy-C₁₋₆ alkyl, wherein said phenyl, furyl, thienyl or cycloalkyl are optionally substituted with 1-3 groups selected from C₁₋₆ alkyl, C₁₋₆ alkyloxy, C₁₋₆alkyloxy-C₁₋₆ alkyl, nitro, halogen, trihalomethyl, amino or hydroxy; or
(v) Furyl, furyloxy, thienyl, thienyloxy, naphthyl, naphthyloxy, phthalanyl or phthalanyloxy optionally substituted with 1-3 groups selected from C₁₋₆ alkyl, C₂₋₆ alkenyl, C₂₋₆ alkynyl, C₁₋₆ akyloxy, C₁₋₆ alkyloxy-C₁₋₆ alkyl, C₁₋₆ alkyloxy-C₁₋₆ alkyloxy, C₂₋₆ alkenyloxy-C₁₋₆ alkyl, C₁₋₆ alkyl substituted with 1-3 groups of hydroxy or halogen, C₁₋₆ alkylthio, C₁₋₆ alkylthio-C₁₋₆ alkyl, C₁₋₆ alkylthio-C₁₋₆ alkyloxy, C₁₋₆ alkenylthio-C₁₋₆ alkyl, C₁₋₆ alkylsulfonyl, halogen, trihalomethyl, cyano, nitro, amino, hydroxy, C₃₋₇ cycloalkyl, C₃₋₇ cycloalkyloxy, C₃₋₇ cycloalkyl-C₁₋₆ alkyl, C₃₋₇ cycloalkyloxy-C₁₋₆ alkyl, phenyl, phenyloxy, phenyl-C₁₋₆ alkyl, phenyl-C₂₋₆ alkenyl, phenyl-C₂₋₆ alkynyl, phenyloxy-C₁₋₆ alkyl, phenyloxy-C₂₋₆ alkenyl, phenyloxy-C₂₋₆ alkynyl, furyl, furyloxy, furyl-C₁₋₆ alkyl, furyloxy-C₁₋₆ alkyl, thienyl, thienyloxy, thienyl-C₁₋₆ alkyl and thienyloxy-C₁₋₆ alkyl, said phenyl, furyl, thienyl or cycloalkyl optionally substituted with 1-3 groups selected from C₁₋₆ alkyl, C₁₋₆ alkyloxy, C₁₋₆alkyloxy-C₁₋₆ alkyl, nitro, halogen, trihalomethyl, amino and hydroxy;
R^{5b} represents hydrogen or C 1-6 alkyl;
R^{1c} represents hydroxy, C₁₋₆ alkyloxy or NR^{6c}R^{7c}, wherin R^{6c} and R^{7c} are each independently hydrogen or C₁₋₆ alkyl;
R^{2c} represents hydrogen or hydroxy;
R^{3c} represents a single bond or C₁₋₆ alkylene;
R^{4c} represents
(i) C₁₋₈ alkyl, C₂₋₈ alkenyl, C₂₋₈ alkynyl, phenyl or cycloalkyl, wherein said alkyl, alkenyl, alkynyl are optionally substituted with phenyl, cycloalkyl, OH, C₁₋₆ alkyloxy or halogen;
(ii) Phenyloxy or C₃₋₇ cycloalkyloxy;
(iii) Furyl, furyloxy, thienyl, thienyloxy, naphthyl, naphthyloxy, phthalanyl or phthalanyloxy;
(iv) Phenyl, phenyloxy, C₃₋₇ cycloalkyl or C₃₋₇ cycloalkyloxy, wherein said phenyl, phenyloxy, cycloalkyl and cycloalkyloxy groups are optionally substituted with 1-3 substituents selected from C₁₋₆ alkyl, C₂₋₆ alkenyl, C₂₋₆ alkynyl, C₁₋₆ alkyloxy, C₁₋₆ alkyloxy-C₁₋₆ alkyl, C₁₋₆ alkyloxy-C₁₋₆ alkyloxy, C₁₋₆ alkenyloxy-C₁₋₆ alkyl, C₁₋₆ alkyl substituted by 1-3 hydroxy or halogen groups, C₁₋₆ alkylthio, C₁₋₆ alkylthio-C₁₋₆ alkyl, C₁₋₆ alkylthio-C₁₋₆ alkyloxy, C₁₋₆ alkenylthio-C₁₋₆ alkyl, C₁₋₆ alkylsulfonyl, halogen, trihalomethyl, cyano, nitro, amino, OH, C₃₋₇ cycloalkyl, C₃₋₇ cycloalkyloxy, C₃₋₇ cycloalkyl-C₁₋₆ alkyl, C₃₋₇ cycloalkyloxy-C₁₋₆ alkyl, phenyl, phenyloxy, phenyl-C₁₋₆ alkyl, phenyl-C₂₋₆ alkenyl, phenyl-C₂₋₆ alkynyl, phenyloxy-C₁₋₆ alkyl, phenyloxy-C₂₋₆ alkenyl, phenyloxy-C₂₋₆ alkynyl, furyl, furyloxy, furyl-C₁₋₆ alkyl, furyloxy-C₁₋₆ alkyl, thienyl, thienyloxy, thienyl-C₁₋₆ alkyl or thienyloxy-C₁₋₆ alkyl, wherein said phenyl, furyl, thienyl or cycloalkyl are optionally substituted with 1-3 groups selected from C₁₋₆ alkyl, C₁₋₆ alkyloxy, C₁₋₆alkyloxy-C₁₋₆ alkyl, nitro, halogen, trihalomethyl, amino or hydroxy; or
(v) Furyl, furyloxy, thienyl, thienyloxy, naphthyl, naphthyloxy, phthalanyl or phthalanyloxy optionally substituted with 1-3 groups selected from C₁₋₆ alkyl, C₂₋₆ alkenyl, C₂₋₆ alkynyl, C₁₋₆ akyloxy, C₁₋₆ alkyloxy-C₁₋₆ alkyl, C₁₋₆ alkyloxy-C₁₋₆ alkyloxy, C₂₋₆ alkenyloxy-C₁₋₆ alkyl, C₁₋₆ alkyl substituted with 1-3 groups of hydroxy or halogen, C₁₋₆ alkylthio, C₁₋₆ alkylthio-C₁₋₆ alkyl, C₁₋₆ alkylthio-C₁₋₆ alkyloxy, C₁₋₆ alkenylthio-C₁₋₆ alkyl, C₁₋₆ alkylsulfonyl, halogen, trihalomethyl, cyano, nitro, amino, hydroxy, C₃₋₇ cycloalkyl, C₃₋₇ cycloalkyloxy, C₃₋₇ cycloalkyl-C₁₋₆ alkyl, C₃₋₇ cycloalkyloxy-C₁₋₆ alkyl, phenyl, phenyloxy, phenyl-C₁₋₆ alkyl, phenyl-C₂₋₆ alkenyl, phenyl-C₂₋₆ alkynyl, phenyloxy-C₁₋₆ alkyl, phenyloxy-C₂₋₆ alkenyl, phenyloxy-C₂₋₆ alkynyl, furyl, furyloxy, furyl-C₁₋₆ alkyl, furyloxy-C₁₋₆ alkyl, thienyl, thienyloxy, thienyl-C₁₋₆ alkyl and thienyloxy-C₁₋₆ alkyl, said phenyl, furyl, thienyl or cycloalkyl optionally substituted with 1-3 groups selected from C₁₋₆ alkyl, C₁₋₆ alkyloxy, C₁₋₆alkyloxy-C₁₋₆ alkyl, nitro, halogen, trihalomethyl, amino and hydroxy;
R^{5c} represents hydrogen, hydroxy or C 1-6 alkyl;
R^{1d} represents hydroxy, C₁₋₆ alkyloxy or NR^{6d}R^{7d}, wherin R^{6d} and R^{7d} are each independently hydrogen or C₁₋₆ alkyl;
R^{2d} represents hydrogen or hydroxy;
R^{3d} represents a single bond or C₁₋₆ alkylene;
R^{4d} represents
(i) C₁₋₈ alkyl, C₂₋₈ alkenyl, C₂₋₈ alkynyl, phenyl or cycloalkyl, wherein said alkyl, alkenyl, alkynyl are optionally substituted with phenyl, cycloalkyl, OH, C₁₋₆ alkyloxy or halogen;
(ii) Phenyloxy or C₃₋₇ cycloalkyloxy;
(iii) Furyl, furyloxy, thienyl, thienyloxy, naphthyl, naphthyloxy, phthalanyl or phthalanyloxy;
(iv) Phenyl, phenyloxy, C₃₋₇ cycloalkyl or C₃₋₇ cycloalkyloxy, wherein said phenyl, phenyloxy, cycloalkyl and cycloalkyloxy groups are optionally substituted with 1-3 substituents selected from C₁₋₆ alkyl, C₂₋₆ alkenyl, C₂₋₆ alkynyl, C₁₋₆ alkyloxy, C₁₋₆ alkyloxy-C₁₋₆ alkyl, C₁₋₆ alkyloxy-C₁₋₆ alkyloxy, C₁₋₆ alkenyloxy-C₁₋₆ alkyl, C₁₋₆ alkyl substituted by 1-3 hydroxy or halogen groups, C₁₋₆ alkylthio, C₁₋₆ alkylthio-C₁₋₆ alkyl, C₁₋₆ alkylthio-C₁₋₆ alkyloxy, C₁₋₆ alkenylthio-C₁₋₆ alkyl, C₁₋₆ alkylsulfonyl, halogen, trihalomethyl, cyano, nitro, amino, OH, C₃₋₇ cycloalkyl, C₃₋₇ cycloalkyloxy, C₃₋₇ cycloalkyl-C₁₋₆ alkyl, C₃₋₇ cycloalkyloxy-C₁₋₆ alkyl, phenyl, phenyloxy, phenyl-C₁₋₆ alkyl, phenyl-C₂₋₆ alkenyl, phenyl-C₂₋₆ alkynyl, phenyloxy-C₁₋₆ alkyl, phenyloxy-C₂₋₆ alkenyl, phenyloxy-C₂₋₆ alkynyl, furyl, furyloxy, furyl-C₁₋₆ alkyl, furyloxy-C₁₋₆ alkyl, thienyl, thienyloxy, thienyl-C₁₋₆ alkyl or thienyloxy-C₁₋₆ alkyl, wherein said phenyl, furyl, thienyl or cycloalkyl are optionally substituted with 1-3 groups selected from C₁₋₆ alkyl, C₁₋₆ alkyloxy, C₁₋₆alkyloxy-C₁₋₆ alkyl, nitro, halogen, trihalomethyl, amino or hydroxy; or
(v) Furyl, furyloxy, thienyl, thienyloxy, naphthyl, naphthyloxy, phthalanyl or phthalanyloxy optionally substituted with 1-3 groups selected from C₁₋₆ alkyl, C₂₋₆ alkenyl, C₂₋₆ alkynyl, C₁₋₆ akyloxy, C₁₋₆ alkyloxy-C₁₋₆ alkyl, C₁₋₆ alkyloxy-C₁₋₆ alkyloxy, C₂₋₆ alkenyloxy-C₁₋₆ alkyl, C₁₋₆ alkyl substituted with 1-3 groups of hydroxy or halogen, C₁₋₆ alkylthio, C₁₋₆ alkylthio-C₁₋₆ alkyl, C₁₋₆ alkylthio-C₁₋₆ alkyloxy, C₁₋₆ alkenylthio-C₁₋₆ alkyl, C₁₋₆ alkylsulfonyl, halogen, trihalomethyl, cyano, nitro, amino, hydroxy, C₃₋₇ cycloalkyl, C₃₋₇ cycloalkyloxy, C₃₋₇ cycloalkyl-C₁₋₆ alkyl, C₃₋₇ cycloalkyloxy-C₁₋₆ alkyl, phenyl, phenyloxy, phenyl-C₁₋₆ alkyl, phenyl-C₂₋₆ alkenyl, phenyl-C₂₋₆ alkynyl, phenyloxy-C₁₋₆ alkyl, phenyloxy-C₂₋₆ alkenyl, phenyloxy-C₂₋₆ alkynyl, furyl, furyloxy, furyl-C₁₋₆ alkyl, furyloxy-C₁₋₆ alkyl, thienyl, thienyloxy, thienyl-C₁₋₆ alkyl and thienyloxy-C₁₋₆ alkyl, said phenyl, furyl, thienyl or cycloalkyl optionally substituted with 1-3 groups selected from C₁₋₆ alkyl, C₁₋₆ alkyloxy, C₁₋₆alkyloxy-C₁₋₆ alkyl, nitro, halogen, trihalomethyl, amino and hydroxy;
R^{5d} represents hydrogen, hydroxy or C 1-6 alkyl;
R^{1e} represents carboxyl, (C₃-C₄)alkoxylcarbonyl or tetrazolyl;
R^{2e} represents —Ar, or —Ar¹-V-Ar², wherein V is a bond, —O—, —OCH₂— or —CH₂O—;
X represents —CH₂— or O;
Z represents —(CH₂)₃—, thienyl, thiazolyl, or phenyl;
Ar represents a partially saturated, fully saturated or fully unsaturated five to eight membered ring optionally having one to four heteroatoms selected independently from oxygen, sulfur and nitrogen, or a bicyclic ring consisting of two fused independently partially saturated, fully saturated or fully unsaturated five or six membered rings, taken independently, optionally having one to four heteroatoms selected independently from nitrogen, sulfur and oxygen, said partially or fully saturated ring or bicyclic ring optionally having one or two oxo groups substituted on carbon or one or two oxo groups substituted on sulfur; and
Ar¹ and Ar² each independently represent a partially saturated, fully saturated or fully unsaturated five to eight membered ring optionally having one to four heteroatoms selected independently from oxygen, sulfur and nitrogen, said partially or fully saturated ring optionally having one or two oxo groups substituted on carbon or one or two oxo groups substituted on sulfur;
wherein said Ar moiety is optionally substituted on carbon or nitrogen, on one ring if the moiety is monocyclic, or on one or both rings if the moiety is bicyclic, with up to three substituents per ring each independently selected from hydroxy, halo, carboxy, (C₁-C₇)alkoxy, (C₁-C₄)alkoxy(C₁-C₄)alkyl, (C₁-C₇)alkyl, (C₂-C₇)alkenyl, (C₃-C₇)cycloalkyl, (C₃-C₇)cycloalkyl(C₁-C₄)alkyl, (C₃-C₇)cycloalkyl(C₁-C₄)alkanoyl, formyl, (C₁-C₈)alkanoyl, (C₁-C₆)alkanoyl(C₁-C₆)alkyl, (C₁-C₄)alkanoylamino, (C₁-C₄)alkoxycarbonylamino, hydroxysulfonyl, aminocarbonylamino or mono-N-, di-N,N-, di-N,N′- or tri-N,N,N′-(C₁-C₄)alkyl substituted aminocarbonylamino, sulfonamido, (C₁-C₄)alkylsulfonamido, amino, mono-N- or di-N,N-(C₁-C₄)alkylamino, carbamoyl, mono-N- or di-N,N-(C₁-C₄)alkylcarbamoyl, cyano, thiol, (C₁-C₆)alkylthio, (C₁-C₆)alkylsulfinyl, (C₁-C₄)alkylsulfonyl and mono-N- or di-N,N-(C₁-C₄)alkylaminosulfinyl, wherein said alkyl and alkoxy substituents in the definition of Ar are optionally substituted on carbon with up to three fluoro; and
wherein said Ar¹ and Ar² moieties are independently optionally substituted on carbon or nitrogen with up to three substituents each independently selected from hydroxy, halo, carboxy, (C₁-C₇)alkoxy, (C₁-C₄)alkoxy(C₁-C₄)alkyl, (C₁-C₇)alkyl, (C₂-C₇)alkenyl, (C₃-C₇)cycloalkyl, (C₃-C₇)cycloalkyl(C₁-C₄)alkyl, (C₃-C₇)cycloalkyl(C₁-C₄)alkanoyl, formyl, (C₁-C₈)alkanoyl, (C₁-C₆)alkanoyl(C₁-C₆)alkyl, (C₁-C₄)alkanoylamino, (C₁-C₄)alkoxycarbonylamino, hydroxysulfonyl, aminocarbonylamino or mono-N-, di-N,N-, di-N,N′- or tri-N,N,N′-(C₁-C₄)alkyl substituted aminocarbonylamino, sulfonamido, (C₁-C₄)alkylsulfonamido, amino, mono-N- or di-N,N-(C₁-C₄)alkylamino, carbamoyl, mono-N- or di-N,N-(C₁-C₄)alkylcarbamoyl, cyano, thiol, (C₁-C₆)alkylthio, (C₁-C₆)alkylsulfinyl, (C₁-C₄)alkylsulfonyl and mono-N- or di-N,N-(C₁-C₄)alkylaminosulfinyl, wherein said alkyl and alkoxy substituents in the definition of Ar¹ and Ar² are optionally substituted on carbon with up to three fluoro.

2. The method according to claim 1 of formula Ia wherein n is 3 and all other variables are as originally described.

3. The method according to claim 2 wherein R₃ and R₄ are each independently hydrogen or hydroxy and all other variables are as originally described.

4. The method according to claim 3 wherein R₁ is COOH all other variables are as originally described.

5. The method according to claim 2 wherein R₁ is tetrazol-5-yl and all other variables are as originally described

6. The method according to claim 5 wherein R₁ is tetrazol-5-yl, R₂ is phenyl and all other variables are as originally described.

7. The method according to claim 3 wherein R₈ is CH₂ and all other variables are as originally described

8. The method according to claim 1 of formula Ib wherein R^{1b} is hydroxy.

9. The method according to claim 1 of formula Ib wherein R^{1b} is C₁₋₆ alkyloxy.

10. The method according to claim 1 of formula Ib wherein R^{1b} is NR^{6b}R^{7b}, wherein R^{6b} and R^{7b} are each independently hydrogen or C₁₋₆ alkyl.

11. The method according to claim 1 of formula Ib wherein R^{3b} is a single bond.

12. The method according to claim 11 wherein R^{4b} is C₁₋₈ alkyl, C₂₋₈ alkenyl, phenyl, C₃₋₇ cycloalkyl or C₂₋₈ alkynyl, wherein said alkyl, alkenyl and alkynyl are optionally substituted with C₁₋₆ alkyloxy or halogen.

13. The method according to claim 12 wherein R^{4b} is C₁₋₈ alkyl, C₂₋₈ alkenyl, or C₂₋₈ alkynyl, wherein said alkyl, alkenyl and alkynyl are optionally substituted with C₁₋₆ alkyloxy or halogen.

14. The method according to claim 11 wherein R^{4b} is phenyloxy or C₃₋₇ cycloalkyloxy.

15. The method according to claim 1 wherein R^{1c} is C₁₋₆ alkyloxy, R^{2c} is hydrogen, R^{5c} is hydroxy, and R^{4c} is phenyl which is optionally substituted with 1-3 substituents selected from C₁₋₆ alkyl, C₂₋₆ alkenyl, C₂₋₆ alkynyl, C₁₋₆ alkyloxy, C₁₋₆ alkyloxy-C₁₋₆ alkyl, C₁₋₆ alkyloxy-C₁₋₆ alkyloxy, C₁₋₆ alkenyloxy-C₁₋₆ alkyl, C₁₋₆ alkyl substituted by 1-3 hydroxy or halogen groups, C₁₋₆ alkylthio, C₁₋₆ alkylthio-C₁₋₆ alkyl, C₁₋₆ alkylthio-C₁₋₆ alkyloxy, C₁₋₆ alkenylthio-C₁₋₆ alkyl, C₁₋₆ alkylsulfonyl, halogen, trihalomethyl, cyano, nitro, amino, OH, C₃₋₇ cycloalkyl, C₃₋₇ cycloalkyloxy, C₃₋₇ cycloalkyl-C₁₋₆ alkyl, C₃₋₇ cycloalkyloxy-C₁₋₆ alkyl, phenyl, phenyloxy, phenyl-C₁₋₆ alkyl, phenyl-C₂₋₆ alkenyl, phenyl-C₂₋₆ alkynyl, phenyloxy-C₁₋₆ alkyl, phenyloxy-C₂₋₆ alkenyl, phenyloxy-C₂₋₆ alkynyl, furyl, furyloxy, furyl-C₁₋₆ alkyl, furyloxy-C₁₋₆ alkyl, thienyl, thienyloxy, thienyl-C₁₋₆ alkyl or thienyloxy-C₁₋₆ alkyl, wherein said phenyl, furyl, thienyl or cycloalkyl are optionally substituted with 1-3 groups selected from C₁₋₆ alkyl, C₁₋₆ alkyloxy, C₁₋₆alkyloxy-C₁₋₆ alkyl, nitro, halogen, trihalomethyl, amino or hydroxy.

16. The method according to claim 1 wherein R^{1d} is C₁₋₆ hydroxy, R^{2d} is hydrogen, R^{5d} is hydroxy, and R^{4d} is phenyl which is optionally substituted with 1-3 substituents selected from C₁₋₆ alkyl, C₂₋₆ alkenyl, C₂₋₆ alkynyl, C₁₋₆ alkyloxy, C₁₋₆ alkyloxy-C₁₋₆ alkyl, C₁₋₆ alkyloxy-C₁₋₆ alkyloxy, C₁₋₆ alkenyloxy-C₁₋₆ alkyl, C₁₋₆ alkyl substituted by 1-3 hydroxy or halogen groups, C₁₋₆ alkylthio, C₁₋₆ alkylthio-C₁₋₆ alkyl, C₁₋₆ alkylthio-C₁₋₆ alkyloxy, C₁₋₆ alkenylthio-C₁₋₆ alkyl, C₁₋₆ alkylsulfonyl, halogen, trihalomethyl, cyano, nitro, amino, OH, C₃₋₇ cycloalkyl, C₃₋₇ cycloalkyloxy, C₃₋₇ cycloalkyl-C₁₋₆ alkyl, C₃₋₇ cycloalkyloxy-C₁₋₆ alkyl, phenyl, phenyloxy, phenyl-C₁₋₆ alkyl, phenyl-C₂₋₆ alkenyl, phenyl-C₂₋₆ alkynyl, phenyloxy-C₁₋₆ alkyl, phenyloxy-C₂₋₆ alkenyl, phenyloxy-C₂₋₆ alkynyl, furyl, furyloxy, furyl-C₁₋₆ alkyl, furyloxy-C₁₋₆ alkyl, thienyl, thienyloxy, thienyl-C₁₋₆ alkyl or thienyloxy-C₁₋₆ alkyl, wherein said phenyl, furyl, thienyl or cycloalkyl are optionally substituted with 1-3 groups selected from C₁₋₆ alkyl, C₁₋₆ alkyloxy, C₁₋₆alkyloxy-C₁₋₆ alkyl, nitro, halogen, trihalomethyl, amino or hydroxy.

17. The method according to claim 1 wherein X is —CH₂—, Z is —(CH₂)₃—, 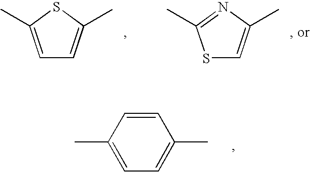
and R^{2e} is Ar.

18. A method for treating disorders related to elevated intraocular pressure by: treating ocular hypertension, treating glaucoma, treating macular edema, treating macular degeneration, increasing retinal and optic nerve head blood velocity, increasing retinal and optic nerve tension, providing a neuroprotective effect or treating dry eyes, comprising administration to a patient in need of such treatment a therapeutically effective amount of a compound of structural formula Ia or Ib: 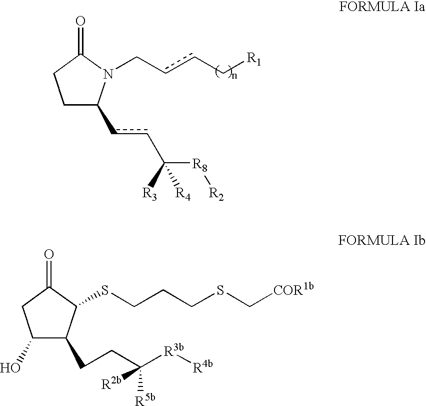
R₁ represents COORs, CONHR₆ or tetrazol-5-yl;
R₃ and R₄ are each independently hydrogen or hydroxy;
R₂ represents a-thienyl, phenyl or phenoxy, wherein said phenyl and phenoxy are optionally substituted with 1-3 substituents selected from chloro, fluoro, phenyl, methoxy, trifluoromethyl or C₁₋₃ alkyl; [symbol]
represents a single or double bond;
n is 0 to 3;
R₅ represents hydrogen, C₁₋₅ alkyl, phenyl or p-biphenyl;
R₆ represents COR₇ or SO₂R₇;
R₇ represents phenyl or C₁₋₅ alkyl;
R₈ represents CH₂;
R^{1b} represents hydroxy, C₁₋₆ alkyloxy or NR^{6b}R^{7b}, wherin R^{6b} and R^{7b} are each independently hydrogen or C₁₋₆ alkyl;
R^{2b} represents hydrogen or hydroxy;
R^{3b} represents a single bond or C₁₋₆ alkylene;
R^{4b} represents
(vi) C₁₋₈ alkyl, C₂₋₈ alkenyl, C₂₋₈ alkynyl, phenyl or cycloalkyl, wherein said alkyl, alkenyl, alkynyl are optionally substituted with phenyl, cycloalkyl, OH, C₁₋₆ alkyloxy or halogen;
(vii) Phenyloxy or C₃₋₇ cycloalkyloxy;
(viii) Furyl, furyloxy, thienyl, thienyloxy, naphthyl, naphthyloxy, phthalanyl or phthalanyloxy;
(ix) Phenyl, phenyloxy, C₃₋₇ cycloalkyl or C₃₋₇ cycloalkyloxy, wherein said phenyl, phenyloxy, cycloalkyl and cycloalkyloxy groups are optionally substituted with 1-3 substituents selected from C₁₋₆ alkyl, C₂₋₆ alkenyl, C₂₋₆ alkynyl, C₁₋₆ alkyloxy, C₁₋₆ alkyloxy-C₁₋₆ alkyl, C₁₋₆ alkyloxy-C₁₋₆ alkyloxy, C₁₋₆ alkenyloxy-C₁₋₆ alkyl, C₁₋₆ alkyl substituted by 1-3 hydroxy or halogen groups, C₁₋₆ alkylthio, C₁₋₆ alkylthio-C₁₋₆ alkyl, C₁₋₆ alkylthio-C₁₋₆ alkyloxy, C₁₋₆ alkenylthio-C₁₋₆ alkyl, C₁₋₆ alkylsulfonyl, halogen, trihalomethyl, cyano, nitro, amino, OH, C₃₋₇ cycloalkyl, C₃₋₇ cycloalkyloxy, C₃₋₇ cycloalkyl-C₁₋₆ alkyl, C₃₋₇ cycloalkyloxy-C₁₋₆ alkyl, phenyl, phenyloxy, phenyl-C₁₋₆ alkyl, phenyl-C₂₋₆ alkenyl, phenyl-C₂₋₆ alkynyl, phenyloxy-C₁₋₆ alkyl, phenyloxy-C₂₋₆ alkenyl, phenyloxy-C₂₋₆ alkynyl, furyl, furyloxy, furyl-C₁₋₆ alkyl, furyloxy-C₁₋₆ alkyl, thienyl, thienyloxy, thienyl-C₁₋₆ alkyl or thienyloxy-C₁₋₆ alkyl, wherein said phenyl, furyl, thienyl or cycloalkyl are optionally substituted with 1-3 groups selected from C₁₋₆ alkyl, C₁₋₆ alkyloxy, C₁₋₆alkyloxy-C₁₋₆ alkyl, nitro, halogen, trihalomethyl, amino or hydroxy; or
(x) Furyl, furyloxy, thienyl, thienyloxy, naphthyl, naphthyloxy, phthalanyl or phthalanyloxy optionally substituted with 1-3 groups selected from C₁₋₆ alkyl, C₂₋₆ alkenyl, C₂₋₆ alkynyl, C₁₋₆ akyloxy, C₁₋₆ alkyloxy-C₁₋₆ alkyl, C₁₋₆ alkyloxy-C₁₋₆ alkyloxy, C₂₋₆ alkenyloxy-C₁₋₆ alkyl, C₁₋₆ alkyl substituted with 1-3 groups of hydroxy or halogen, C₁₋₆ alkylthio, C₁₋₆ alkylthio-C₁₋₆ alkyl, C₁₋₆ alkylthio-C₁₋₆ alkyloxy, C₁₋₆ alkenylthio-C₁₋₆ alkyl, C₁₋₆ alkylsulfonyl, halogen, trihalomethyl, cyano, nitro, amino, hydroxy, C₃₋₇ cycloalkyl, C₃₋₇ cycloalkyloxy, C₃₋₇ cycloalkyl-C₁₋₆ alkyl, C₃₋₇ cycloalkyloxy-C₁₋₆ alkyl, phenyl, phenyloxy, phenyl-C₁₋₆ alkyl, phenyl-C₂₋₆ alkenyl, phenyl-C₂₋₆ alkynyl, phenyloxy-C₁₋₆ alkyl, phenyloxy-C₂₋₆ alkenyl, phenyloxy-C₂₋₆ alkynyl, furyl, furyloxy, furyl-C₁₋₆ alkyl, furyloxy-C₁₋₆ alkyl, thienyl, thienyloxy, thienyl-C₁₋₆ alkyl and thienyloxy-C₁₋₆ alkyl, said phenyl, furyl, thienyl or cycloalkyl optionally substituted with 1-3 groups selected from C₁₋₆ alkyl, C₁₋₆ alkyloxy, C₁₋₆alkyloxy-C₁₋₆ alkyl, nitro, halogen, trihalomethyl, amino and hydroxy;
R^{5b} represents hydrogen or C 1-6 alkyl.

19. The method according to claim 1 wherein the compound is:
7-(2S-[3R-hydroxy-4-(3-phenoxy-phenyl)-butyl]-5-oxo-pyrrolidin-1-yl)-heptanoic acid;
7-(2S-[3R-hydroxy-4-(3-trifluoromethyl-phenyl)-butyl]-5-oxo-pyrrolidin-1-yl)-heptanoic acid;
7-(2S-[4-(3-chloro-phenyl)-3R-hydroxy-butyl]-5-oxo-pyrrolidin-1-yl)-heptanoic acid;
7-(2S-[3R-hydroxy-4-phenyl-butyl]-5-oxo-pyrrolidin-1-yl)-heptanoic acid;
7-(2R-[3S-hydroxy-4-phenyl-but-1-enyl]-5-oxo-pyrrolidin-1-yl)-heptanoic acid;
5S-(4-(3-chloro-phenyl)-3R-hydroxy-butyl)-1-(6-(2H-tetrazol-5-yl)-hexyl-pyrrolidin-2-one;
5S-(3R-hydroxy-4-(3-trifluoromethyl-phenyl)-butyl)-1-(6-(2H-tetrazol-5-yl)-hexyl-pyrrolidin-2-one;
5S-(3R-hydroxy-4-phenyl-butyl)-1-[6-(1H-tetrazol-5-yl)-hexyl]-pyrrolidin-2-one;
5R-(3S-hydroxy-4-phenyl-but-1-enyl)-1-[6-(1H-tetrazol-5-yl)-hexyl]-pyrrolidin-2-one;
7-{(2R)-2-[(1E)-3-hydroxy-4-methyl-4-phenylpent-1-enyl]-5-oxopyrrolidin-1-yl}heptanoic acid;
7-{(2R)-2-[(1E)-3-hydroxy-3-(1-phenylcyclopropyl)prop-1-enyl]-5-oxopyrrolidin-1-yl}heptanoic acid;
7-{(2R)-2-[(1E)-3-cyclohexyl-3-hydroxyprop-1-enyl]-5-oxopyrrolidin-1-yl}heptanoic acid;
11α,15α-dihydroxy-9-oxo-16-(3-methoxymethylphenyl)-17,18,19,20-tetranor-3,7-dithiaprost-13E-enoic acid or methyl ester thereof;
11α,15α-dihydroxy-9-oxo-16-(5-methoxymethylthiophen-2-yl)-17,18,19,20-tetranor-3,7-dithiaprost-13E-enoic acid or methyl ester thereof;
11α,15α-dihydroxy-9-oxo-16-phenyloxy-17,18,19,20-tetranor-3,7-dithiaprost-13E-enoic acid or methyl ester thereof;
11α,15α-dihydroxy-9-oxo-16-(4-methylphenyl)-17,18,19,20-tetranor-3,7-dithiaprost-13E-enoic acid or methyl ester thereof;
11α,15α-dihydroxy-9-oxo-16-(4-chlorophenyl)-17,18,19,20-tetranor-3,7-dithiaprost-13E-enoic acid or methyl ester thereof;
11α,15α-dihydroxy-9-oxo-16-(3-thienyl)-17,18,19,20-tetranor-3,7-dithiaprost-13E-enoic acid or methyl ester thereof;
11α,15α-dihydroxy-9-oxo-16-(2-naphthyl)-17,18,19,20-tetranor-3,7-dithiaprost-13E-enoic acid or methyl ester thereof;
11α,15α-dihydroxy-9-oxo-16-(5-phthalanyl)-17,18,19,20-tetranor-3,7-dithiaprost-13E-enoic acid or methyl ester thereof;
11α,15α-dihydroxy-9-oxo-16-(4-methoxyphenyl)-17,18,19,20-tetranor-3,7-dithiaprost-13E-enoic acid or methyl ester thereof;
11α,15α-dihydroxy-9-oxo-16-(4-methoxy-3-chlorophenyl)-17,18,19,20-tetranor-3,7-dithiaprost-13E-enoic acid;
11α,15α-dihydroxy-9-oxo-16-(3-trifluoromethylphenyl)-17,18,19,20-tetranor-3,7-dithiaprost-13E-enoic acid;
11α,15α-dihydroxy-9-oxo-16-phenyl-17,18,19,20-tetranor-3,7-dithiaprost-13E-enoic acid;
11α,15α-dihydroxy-9-oxo-17-phenyl-17,18,19,20-tetranor-3,7-dithiaprost-13E-enoic acid;
11α,15α-dihydroxy-9-oxo-16α-methyl-16-phenyl-3,7-dithia-20-norprost-13E-enoic acid or methyl ester thereof;
11α,15α-dihydroxy-9-oxo-16-cyclohexyl-3,7-dithia-17,18,19,20-tetranorprost-13E-enoic acid;
11α,15α-dihydroxy-9-oxo-19,20-methano-3,7-dithiaprost-13E-enoic acid;
11α,15α-dihydroxy-9-oxo-16-cyclopentyll-3,7-dithia-17,18,19,20-tetranorprost-13E-enoic acid or methyl ester thereof;
11α,15α-dihydroxy-9-oxo-15-cyclohexyl-3,7-dithia-16,17,18,19,20-pentanorprost-13E-enoic acid or methyl ester thereof;
(11α,13E,15α)-9-oxo-11,15-dihydroxy-16-(3-methoxymethylphenyl)-17,18,19,20-tetranor-5-thiaprost-13-enoic acid, or methyl, n-propyl, i-propyl or n-butyl ester thereof;
(11α,13E, 15α)-9-oxo-11,15-dihydroxy-16-(3-ethoxymethylphenyl)-17,18,19,20-tetranor-5-thiaprost-13-enoic acid, or methyl or ethyl ester thereof;
(11α,13E,15α)-9-oxo-11,15-dihydroxy-16-(3-n-propyloxymethylphenyl)-17,18,19,20-tetranor-5-thiaprost-13-enoic acid, or methyl or t-butyl ester thereof;
(11α,15α)-9-oxo-11,15-dihydroxy-16-(3-methoxymethylphenyl)-17,18,19,20-tetranor-5-thiaprostanoic acid or methyl ester thereof;
(11α,15α,13E)-9-oxo-11,15-dihydroxy-16-methyl-16-(3-methoxymethylphenyl)-17,18,19,20-tetranor-5-thiaprost-13-enoic acid or methyl ester thereof;
(15α,13E)-9-oxo-15-dihydroxy-16-3-methoxymethylphenyl)-17,18,19,20-tetranor-5-thiaprost-13-enoic acid or methyl ester thereof;
(11α,13E,15α)-9-oxo-11,15-dihydroxy-16-(3-methyl-4-hydroxyphenyl)-17,18,19,20-tetranor-5-thiaprost-13-enoic acid or methyl ester thereof;
(11α,15α,13E)-9-oxo-11,15-dihydroxy-16-(3-methyl-4-hydroxyphenyl)-17,18,19,20-tetranor-5-thiaprost-13-enoic acid or methyl ester thereof, or
3S-(3-hydroxy-4-phenylbutyl)-2R-[6-(1H-tetrazol-5-yl)-hexyl]cyclopentanone.

20. The method according to claim 19 wherein the compound is selected from:
7-{(2R)-2-[(1E)-3-hydroxy-3-(1-phenylcyclopropyl)prop-1-enyl]-5-oxopyrrolidin-1-yl}heptanoic acid;
7-{(2R)-2-[(1E)-3-cyclohexyl-3-hydroxyprop-1-enyl]-5-oxopyrrolidin-1-yl}heptanoic acid; and
7-{(2R)-2-[(1E)-3-hydroxy-3-methyl-4-phenylbut-1-enyl]-5-oxopyrrolidin-1-yl}heptanoic acid or its pharmaceutically acceptable salt or ester thereof.

21. The method according to claim 1 wherein the compound of formula Ia, Ib, Ic, Id, Ie or If is applied as a topical formulation.

22. The method according to claim 21 wherein the topical formulation optionally contains xanthan gum or gellan gum.

23. The method according to claim 22 wherein the topical formulation is a solution or suspension.

24. The method according to claim 1 wherein an active ingredient belonging to the group consisting of: β-adrenergic blocking agent, parasympatho-mimetic agent, sympathomimetic agent, carbonic anhydrase inhibitor, and a prostaglandin, hypotensive lipid, neuroprotectant, and 5-HT2 receptor agonist is added to the formulation.

25. The method according to claim 24 wherein the β-adrenergic blocking agent is timolol, betaxolol, levobetaxolol, carteolol, or levobunolol; the parasympathomimetic agent is pilocarpine; the sympathomimetic agent is epinephrine, brimonidine, iopidine, clonidine, or para-aminoclonidine; the carbonic anhydrase inhibitor is dorzolamide, acetazolamide, metazolamide or brinzolamide; the prostaglandin is latanoprost, travaprost, unoprostone, rescula, or S1033; the hypotensive lipid is lumigan; the neuroprotectant is eliprodil, R-eliprodil or memantine; and the 5-HT2 receptor agonist is 1-(2-aminopropyl)-3-methyl-1H-imdazol-6-ol fumarate or 2-(3-chloro-6-methoxy-indazol-1-yl)-1-methyl-ethylamine.
